(19) United States
(12) Patent Application Publication
(10) Pub. No.: US 2026/0335091 A1
(43) Pub. Date: Oct. 29, 2026

(54) ADJUSTABLE IMPLANTS

(71) Applicant: VisageX Medical Ltd., D.N. Misgav (IL)

(72) Inventors: Gideon MEYER-BRODNITZ, Ramot-Menashe (IL); Shahar HARARI, Tel Aviv (IL); Ebrahim ELAHI, New York, NY (US); Evgeny KAPILOV, Karmiel (IL); Leo R. MINDICK, Great Neck, NY (US)

(73) Assignee: VisageX Medical Ltd., D.N. Misgav (IL)

(21) Appl. No.: 19/113,485

(22) PCT Filed: Sep. 20, 2023
(86) PCT No.: PCT/IL2023/051021

§ 371 Date: Mar. 20, 2025

Related U.S. Application Data
(60) Provisional application No. 63/408,120, filed on Sep. 20, 2022.

(51) Int. Cl. A61F 2/30; A61F 2/00

(52) U.S. Cl. CPC A61F 2/30942; A61F 2/0059; A61F 2002/30092; A61F 2002/30291; A61F 2002/30556; A61F 2002/30579; A61F 2002/30668; A61F 2210/0085; A61F 2310/00023

(57) ABSTRACT

A method for shaping tissue, including:
providing an implant comprising at least one actuator configured to expand when exposed to an external energy;
implanting the implant into an implantation site in a patient body between at least one first tissue and at least one second tissue
selectively energizing the at least one actuator;
shaping the at least one first tissue and/or the at least one second tissue according to expansion of the energized at least one actuator.

### RELATED APPLICATION/S

This application claims the benefit of priority under 35 USC § 119(e) of U.S. Provisional Patent Application No. 63/408,120 filed Sep. 20, 2022, the contents of which are incorporated herein by reference in their entirety.

### FIELD AND BACKGROUND OF THE INVENTION

The present invention, in some embodiments thereof, relates to shaping tissue and, more particularly, but not exclusively, to shaping tissue using adjustable implants.

U.S. Pat. No. 7,722,670B2 describes “according to an embodiment of the present invention, an implant device may be provided. The implant may be adapted to manipulate the position of an eyeball associated with a patient, whereby the device comprises an insertion device including a first and a second portion. The first portion may include a first thickness and may be adapted to elevate the position of the eyeball. The second portion may include a second thickness and/or a second position relative the first portion for moving the position of the eyeball in a forward direction” (Abstract).

U.S. Pat. No. 10,779,926B2 describes “a facial implant device includes a support structure adapted to be coupled to a bone surface within a facial region, an outer structure adapted to contour to a predefined anatomy associated with the facial region, and an intermediary bladder structure located between the support structure and the outer structure. The intermediary bladder structure includes an internal volume that is adjusted in response to adapting the outer structure to contour to the predefined anatomy associated with the facial region” (Abstract).

U.S. Pat. No. 9,572,676B2 describes “an inflatable balloon for diagnostics and treatment of diseased discs or fractured bones. The balloon is a multi-volume balloon comprised of a plurality of adjustable and expandable single volumes. Further disclosed are methods of forming, expanding, and implanting the multi-volume balloon for proper placement and stabilization of the diseased discs or fractured bones. Still further disclosed are kits for aligning and stabilizing a bone, disc, or spinal motion segment” (Abstract).

U.S. Patent Application Publication No. US20070067041A1 describes “an inflatable facial implant includes a base having a first side and a second side. A bladder wall is secured to the first side of the base. The bladder wall is more flexible than the base. Surgical methods of using such an implant are also disclosed” (Abstract).

U.S. Patent Application Publication No. US20100249946A1 describes “disclosed are implantable tissue augmentation devices, methods, and associated tools. The devices include an inflatable body, having a self-sealing membrane operably attached to a wall of the implant. The self-sealing membrane provides access for filling the device, and includes a first layer comprising a fabric. The fabric has a first plurality of yarn strands positioned in a first direction, and a second plurality of yarn strands positioned in a second direction. The first and second plurality of yarn strands intersect to form a matrix pattern with cells defined by free spaces between yarn strands. The membrane also includes a first elastomeric material configured to fill the cells as well as form a coating over the first and second plurality of yarn strand, and a second layer comprising a second elastomeric material. The second elastomeric material has a lower durometer than the first elastomeric material. Kits and systems are also disclosed” (Abstract).

Additional background art includes U.S. Patent Application Publication No. US20210369460A1, U.S. Patent Application Publication No. US20140128476A1, U.S. Pat. No. 11,090,149B2, and U.S. Patent Application Publication No. US20170296243A1.

### SUMMARY OF THE INVENTION

Some examples of some embodiments of the invention are listed below (an embodiment may include features from more than one example and/or fewer than all features of an example):
Example 1. A method for shaping tissue, comprising:
providing an implant comprising at least one actuator configured to expand when exposed to an external energy;
implanting the implant into an implantation site in a patient body between at least one first tissue and at least one second tissue
selectively energizing the at least one actuator;
shaping said at least one first tissue and/or said at least one second tissue according to expansion of said energized at least one actuator.
Example 2. A method according to example 1, wherein said selectively energizing comprises remotely selectively energizing said at least one actuator from a remote location outside the body.
Example 3. A method according to any one of examples 1 or 2, wherein said selectively energizing comprises selectively heating said at least one actuator to a temperature level that causes said at least one actuator to expand.
Example 4. A method according to example 3, wherein said selectively heating comprises selectively heating said at least one actuator using induction heating.
Example 5. A method according to any one of examples 3 or 4, wherein said selectively heating comprises exposing said at least one actuator to an electromagnetic field generated outside the body.
Example 6. A method according to example 3, wherein said selectively heating comprises exposing said at least one actuator to at least one of, ultrasound energy, radiofrequency energy, laser and infra-red.
Example 7. A method according to any one of the previous examples, wherein said selectively energizing is performed during said implanting.
Example 8. A method according to any one of the previous examples, comprising allowing said implantation site to heal prior to said selectively energizing.
Example 9. A method according to any one of the previous examples, comprising repeating said selectively energizing and said shaping if said shaped tissue does not acquire a target shape.
Example 10. A method according to any one of the previous examples, wherein said providing comprises providing said implant with at least one tissue interface, and wherein said at least one actuator is coupled to said at least one tissue interface, and wherein said implanting comprises placing in contact said at least one actuator with said at least one second tissue and placing in contact said at least one tissue interface with said at least one first tissue.
Example 11. A method according to any one of the previous examples, wherein said implanting comprises plastic or elastic bending said implant to conform to a surface of said at least one first tissue or to a surface of said at least one second tissue.
Example 12. A method according to any one of the previous examples comprising selecting an implant having a target shape and/or size that fits said implantation site prior to said providing.
Example 13. A method according to any one of the previous examples, comprising modifying a shape and/or size of said provided implant to fit said implantation site, prior to and/or during said implanting.
Example 14. A method according to example 13, wherein said modifying comprises changing a number of actuators of said implant.
Example 15. A method according to any one of the previous examples, wherein said at least one first tissue comprises soft tissue, and wherein said at least one second tissue comprises bone tissue, and wherein said shaping comprises shaping said soft tissue according to expansion of said energized at least one actuator.
Example 16. A method according to any one of examples 1 to 14, wherein said at least one first tissue comprises a first soft tissue, and wherein said at least one second tissue comprises a second soft tissue, and wherein said shaping comprises shaping said first soft tissue according to expansion of said energized at least one actuator.
Example 17. A method according to any one of the previous examples, wherein said at least one actuator comprises a plurality of actuators, and wherein said selectively energizing comprises selectively energizing at least one actuator of said plurality of actuators.
Example 18. A body implant, comprising:
at least one tissue interface configured to be placed in contact with a body tissue;
a plurality of actuators coupled to said at least one tissue interface, wherein at least one actuator of said plurality of actuators is configured to expand and/or to contract when exposed to external energy.
Example 19. A body implant according to example 18, wherein said at least one tissue interface and/or said plurality of actuators are configured to flex.
Example 20. A body implant according to any one of examples 18 or 19, wherein at least some of said plurality of actuators are coupled to each other by one or more connectors.
Example 21. A body implant according to any one of examples 18 to 20, wherein said plurality of actuators are configured to move laterally relative to each other when heated by said external energy.
Example 22. A body implant according to any one of examples 18 to 21, wherein each of said plurality of actuators comprises a shape memory material, configured to expand when heated by said external energy.
Example 23. A body implant according to example 22, wherein said shape memory material comprises a shape memory alloy or a shape memory polymer.
Example 24. A body implant according to any one of examples 22 or 23, wherein each of said plurality of actuators comprises a spring formed from said shape memory material, that is configured to expand when heated by said external energy.
Example 25. A body implant according to example 24, wherein said spring is shaped as a spiral, or a helix.
Example 26. A body implant according to any one of examples 24 or 25, wherein each actuator comprises a base to which said spring is coupled, and wherein bases of of two or more actuators are connected together to form an array of actuators coupled to said at least one interface.
Example 27. A body implant according to any one of examples 18 to 26, wherein said at least one tissue interface comprises at least one of soft and/or flexible portion configured to contact body tissue.
Example 28. A body implant according to any one of examples 18 to 27, wherein said at least one tissue interface comprises at least one first surface configured to contact soft body tissue, and at least one second surface configured to be coupled to said plurality of actuators.
Example 29. A body implant according to example 28, wherein said at least one first surface is soft and/or is flexible.
Example 30. A body implant according to any one of examples 28 or 29, wherein said at least one tissue interface and/or said at least one first surface comprises at least one inflatable chamber. Example 31. A body implant according to any one of examples 28 or 29, wherein said at least one tissue interface comprises at least one chamber filled with fluid or gel.
Example 32. A body implant according to any one of examples 18 to 31, wherein each of said plurality of actuators comprises a separate actuator cover isolating each actuator from other actuators of said plurality of actuators.
Example 33. A body implant according to example 32, wherein said actuator cover comprises a bellow cover, configured to move between a folded state to an unfolded state when said actuator expands.
Example 34. A body implant according to any one of examples 32 or 33, wherein said actuator cover comprises perforations which are shaped and sized to allow tissue ingrowth through said cover into said actuator.
Example 35. A body implant according to example 34, wherein a width of said perforations is in a range between 0.1 mm and 3 mm.
Example 36. A body implant according to any one of examples 18 to 35, wherein each of the actuators comprises a first end coupled to said at least one tissue interface and a second opposite end, and wherein said implant comprises tissue contacting pads each is coupled to said second opposite end of said actuator, and wherein said tissue contacting pads are configured to contact bone tissue.
Example 37. A body implant according to any one of examples 18 to 31, comprising a base, and an implant cover coupled between said at least one tissue interface and said base, wherein said implant cover surrounds said implant and defines an implant inner lumen, and wherein said plurality of actuators are positioned inside said inner lumen between said at least one tissue interface and said base.
Example 38. A body implant according to example 37, wherein said implant cover comprises folds and is configured to move between a folded state to an unfolded state when at least one actuators of said plurality of actuators expands.
Example 39. A body implant according to any one of examples 37 or 38, wherein said implant cover comprises perforations which are shaped and sized to allow tissue ingrowth into said implant.
Example 40. A body implant according to any one of examples 37 to 39, wherein said implant cover is integrated with said at least one tissue interface.
Example 41. A body implant according to any one of examples 37 to 40, comprising a filler within said inner lumen, wherein said filler is configured to expand when said at least one actuator of said plurality of actuators expands.
Example 42. A body implant according to any one of examples 18 to 41, wherein said at least one tissue interface comprises at least one silicon filled compartment.
Example 43. A body implant according to any one of examples 18 to 42, wherein said body implant is configured to move between a collapsed state to an expanded state when said at least one actuator expands, and wherein a thickness of said body implant in said collapsed state is within a range between 1 mm and 4 mm.
Example 44. An inflatable actuator, comprising:
at least one flexible tissue interface configured to contact tissue;
at least one base;
at least one inflatable cell coupled between said at least one base and said at least one flexible tissue interface;
at least one inflation port in said at least one inflatable cell, wherein said at least one inflatable cell is configured to expand when inflated via said at least one inflation port.
Example 45. An inflatable actuator according to example 44, wherein said at least one flexible tissue interface comprises silicon.
Example 46. An inflatable actuator according to any one of examples 44 or 45, wherein said at least one tissue interface is shaped as a dome.
Example 47. An inflatable actuator according to any one of examples 44 to 46, wherein said inflation port is fluidically coupled to an inflation channel passes through said at least one flexible tissue interface.
Example 48. A body implant comprising:
a plurality of inflatable actuators according to example 44, coupled to each other by at least one connector.
Example 49. A body implant according to example 48, comprising at least one inflation channel having at least one inflation port, and wherein inflatable cells of said plurality of inflatable actuators are fluidically coupled to said at least one inflation channel.
Example 50. A body implant comprising:
a base, comprising at least one fluid channel;
a plurality of inflatable actuators coupled to said base;
wherein each of said plurality of inflatable actuators comprise:
   at least one inflatable cell configured to move between a deflated state and an expanded inflated state;
   at least one inflation port fluidically coupled to said at least one inflatable cell;
wherein coupling of each of said plurality of inflatable actuators to said base fluidically connects said inflation port to said at least one fluid channel of said base.
Example 51. A body implant according to example 50, wherein said plurality of inflatable actuators contact each other in an array arrangement on said base.
Example 52. A body implant according to example 50, wherein said plurality of inflatable actuators are spaced apart.
Example 53. A body implant according to any one of examples 50 to 52, wherein each of said inflatable actuators comprises at least one fastener, configured to fasten each inflatable actuator to said base.
Example 54. A body implant according to any one of examples 50 to 53, wherein each of said inflatable actuators comprises at least one syringe coupled to said inflation port, wherein penetration of said syringe into said at least one fluid channel in said base, fluidically connects said at least one inflatable cells with said at least one fluid channel.
Example 55. A body implant, comprising:
an array of actuators formed from shape memory alloy, wherein said actuators are interconnected by shape memory alloy bridges;
wherein at least one actuator of said actuators is configured to expand and contract, and to move laterally relative to other actuators in said array when heated.
Example 56. A body implant according to example 55, wherein said actuators comprise springs formed from said shape memory alloy.
Example 57. A body implant according to any one of examples 55 or 56, at least one tissue interface having at least one first tissue contacting surface and at least one second surface.
Example 58. A body implant according to example 57, wherein said at least one tissue interface comprises at least one chamber filled with fluid or gel.
Example 59. A multi-unit body implant, comprising:
a plurality of single unit implants coupled to each other, wherein each single unit implant comprises:
at least one tissue interface configured to be placed in contact with a body tissue;
at least one expandable actuator coupled to said at least one tissue interface;
at least one connector configured to connect each single unit implant to at least one different single unit implant of said plurality of single unit implants.
Example 60. An implant according to example 59, wherein said at least one first tissue interface is part of said at least one expandable actuator.
Example 61. An implant according to any one of examples 59 or 60, wherein said at least one expandable actuator comprises at least one inflatable chamber, and wherein said at least one expandable actuator is configured to expand when said at least one inflatable chamber is inflated.
Example 62. An implant according to any one of examples 59 or 60, wherein said at least one expandable actuator is formed from shape memory alloy, and is configured to expand when heated above a predetermined temperature level.
Example 63. An implant according to any one of examples 59 to 62, wherein said at least one connector comprises a hinge portion configured to allow movement of a first single unit implant relative to at least one second single unit implant connected to said first single unit implant.
Example 64. An implant according to any one of examples 59 to 63, wherein each single unit implant comprises a base coupled to said at least one expandable actuator opposite to said at least one tissue interface, wherein said base is configured to fixedly couple said single unit implant to a tissue.
Example 65. An implant according to example 64, wherein said base comprises one or more openings which are shaped and sized to allow passage of a screw or a nail through said base and into said tissue to couple said single unit implant to said tissue.
Example 66. A body implant, comprising:
at least one implant cover having a tissue contacting surface and at least one opposite surface, wherein said at least one implant cover comprises at least one central portion, at least one edge portion configured to couple said body implant to tissue, and at least one hinge portion therebetween;
at least one expandable actuator contacting said at least one opposite surface of said at least one central portion, wherein said at least one expandable actuator is configured to move from a collapsed state to an expanded state;
wherein when said at least one expandable actuator expands, said at least one expandable actuator pushes said at least one central portion relative to said at least one edge portion using said hinge portion to acquire a continuous tissue contacting surface of said at least one implant cover.

Some additional examples of some embodiments of the invention are listed below (an embodiment may include features from more than one example and/or fewer than all features of an example):
Example 1. A method for shaping tissue, comprising:
providing an implant comprising at least one actuator configured to expand when exposed to an external energy;
implanting the implant into an implantation site in a patient body between at least one first tissue and at least one second tissue of the body;
selectively energizing the at least one actuator;
shaping said at least one first tissue and/or said at least one second tissue according to expansion of said energized at least one actuator in response to said selectively energizing.
Example 2. A method according to example 1, wherein said selectively energizing comprises remotely selectively energizing said at least one actuator from a remote location outside the body.
Example 3. A method according to any one of examples 1 or 2, wherein said selectively energizing comprises selectively heating said at least one actuator to a temperature level that causes said at least one actuator to expand.
Example 4. A method according to example 3, wherein said selectively heating comprises exposing said at least one actuator to an electromagnetic field generated outside the body.
Example 5. A method according to example 3, wherein said selectively heating comprises exposing said at least one actuator to at least one of, ultrasound energy, radiofrequency energy, laser, infra-red, or warm liquid.
Example 6. A method according to any one of the previous examples, wherein said selectively energizing is performed prior or during said implanting.
Example 7. A method according to any one of the previous examples, comprising allowing said implantation site to heal prior to said selectively energizing.
Example 8. A method according to any one of the previous examples, comprising repeating said selectively energizing and said shaping if said shaped tissue does not acquire a target shape. Example 9. A method according to any one of the previous examples, wherein said providing comprises providing said implant with at least one tissue interface, and wherein said at least one actuator is coupled to said at least one tissue interface, and wherein said implanting comprises placing in contact said at least one actuator with said at least one second tissue and placing in contact said at least one tissue interface with said at least one first tissue.
Example 10. A method according to any one of the previous examples, wherein said implanting comprises plastic or elastic bending said implant to conform to a surface of said at least one first tissue or to a surface of said at least one second tissue.
Example 11. A method according to any one of the previous examples, comprising modifying a shape and/or size of said provided implant to fit said implantation site, prior to and/or during said implanting.
Example 12. A method according to example 11, wherein said modifying comprises changing a number of actuators of said implant.
Example 13. A method according to any one of the previous examples, wherein said at least one first tissue comprises soft tissue, and wherein said at least one second tissue comprises bone tissue, and wherein said shaping comprises shaping said soft tissue according to expansion of said energized at least one actuator.
Example 14. A method according to any one of examples 1 to 12, wherein said at least one first tissue comprises a first soft tissue, and wherein said at least one second tissue comprises a second soft tissue, and wherein said shaping comprises shaping said first soft tissue according to expansion of said energized at least one actuator.
Example 15. A method according to any one of the previous examples, wherein said at least one actuator comprises a plurality of actuators, and wherein said selectively energizing comprises selectively energizing at least one actuator of said plurality of actuators.
Example 16. A body implant configured to be implanted within an implantation site in a body, comprising:
at least one actuator positioning base comprising a plurality of spaced-apart actuators coupling regions each is configured to couple at least one actuator to the positioning base;
a plurality of actuators coupled to said at least one actuator positioning base, wherein at least one actuator of said plurality of actuators is configured to expand and/or to contract when exposed to energy.
Example 17. A body implant according to example 16, wherein said plurality of actuators comprise apertured actuators.
Example 18. A body implant according to any one of examples 16 or 17, wherein said at least one actuator positioning base is configured to flex.
Example 19. A body implant according to any one of examples 16 to 18, wherein said actuator positioning base comprises at least one opening in each of said plurality of spaced-apart actuators coupling regions, configured to couple at least one actuator of said plurality of actuators by a snap connection to said actuator positioning base.
Example 20. A body implant according to any one of examples 16 to 19, wherein at least some of said plurality of actuators are coupled to each other by one or more connectors.
Example 21. A body implant according to any one of examples 16 to 20, wherein said plurality of actuators are configured to move laterally relative to each other when heated by said energy.
Example 22. A body implant according to any one of examples 16 to 21, wherein each of said plurality of actuators comprises a shape memory material, configured to expand when heated by said energy.
Example 23. A body implant according to example 22, wherein at least one actuator of said plurality of actuators is configured to expand at a direction substantially perpendicular to said actuators positioning base, when heated.
Example 24. A body implant according to example 22, wherein said at least one actuator of said plurality of actuators is configured to expand at a direction oriented at an angle between 10 degrees and 170 degrees relative to said actuators positioning base, when heated.
Example 25. A body implant according to any one of examples 22 to 24, wherein each of said plurality of actuators comprises a spring formed from said shape memory material, that is configured to expand when heated by said energy.
Example 26. A body implant according to example 25, wherein said spring is shaped as a spiral, or a helix.
Example 27. A body implant according to any one of examples 25 or 26, wherein each actuator comprises a base to which said spring is coupled, and wherein bases of two or more actuators are connected together to form an array of actuators coupled to said actuator positioning base.
Example 28. A body implant according to any one of examples 16 to 27, wherein said actuator positioning base comprises a tissue interface having at least one of soft and/or flexible portion configured to contact body tissue.
Example 29. A body implant according to example 28, wherein said tissue interface comprises at least one first surface configured to contact soft body tissue, and at least one second surface configured to be coupled to said plurality of actuators, wherein said at least one first surface is soft and/or is flexible.
Example 30. A body implant according to example 29, wherein said at least one tissue interface and/or said at least one first surface comprises at least one inflatable chamber.
Example 31. A body implant according to example 29, wherein said at least one tissue interface comprises at least one chamber filled with fluid or gel.
Example 32. A body implant according to any one of examples 16 to 31, wherein each of said plurality of actuators comprises a separate actuator cover isolating each actuator from other actuators of said plurality of actuators.
Example 33. A body implant according to any one of examples 16 to 32, comprising a flexible cover coupled to said actuators positioning base, wherein said plurality of actuators are positioned inside an inner lumen between said flexible cover and said base.
Example 34. A body implant according to any one of examples 16 to 33, wherein said at least one actuators positioning base and said plurality of actuators form an array of actuators, and wherein said body implant comprises a flexible enclosed cover defining an inner lumen and having an outer surface configured to contact body tissue and an inner surface, and wherein said array of actuators in positioned within said inner lumen and is coupled to said inner surface of said flexible cover.
Example 35. A body implant according to any one of examples 33 or 34, wherein said flexible cover comprises one or more perforations which are shaped and sized to allow tissue ingrowth into said implant and/or injection of fluid into said inner lumen.
Example 36. A body implant according to any one of examples 16 to 35, wherein each of the actuators comprises a first end coupled to said at least one actuator positioning base and a second opposite end, and wherein said implant comprises tissue contacting pads each is coupled to said second opposite end of said actuator, and wherein said tissue contacting pads are configured to contact bone tissue or soft tissue.
Example 37. A body implant according to any one of examples 16 to 36, wherein said body implant is configured to move between a collapsed state to an expanded state when said at least one actuator expands, and wherein a thickness of said body implant in said collapsed state is within a range between 1 mm and 4 mm.
Example 38. A body implant, comprising:
an array of apertured actuators, wherein each actuator is configured to expand when heated;
a cover having a tissue contacting outer surface, wherein said cover surrounds said array of apertured actuators.
Example 39. A body implant according to example 38, wherein said apertured actuators are formed form a shape memory alloy configured to expand and apply force on an inner surface of said cover when said shape memory alloy expands.
Example 40. A body implant according to any one of examples 38 or 39, wherein said apertured actuators are interconnected in said array.
Example 41. A body implant according to any one of examples 38 to 40, wherein said array and said apertured actuators are formed as a single unit.
Example 42. A body implant according to example 41, wherein said array and said apertured actuators are formed as a single unit from a shape memory alloy.
Example 43. A body implant according to any one of examples 38 to 42, wherein said cover forms a pocket surrounding said array of apertured actuators.
Example 44. A body implant according to any one of examples 38 to 43, wherein an apertured actuator of said apertured actuators comprises openings crossing through a body of said apertured actuator.
Example 45. A body implant according to example 44, wherein said apertured actuator is shaped as an extendable spring.
Example 46. An inflatable actuator unit, comprising:
at least one flexible tissue interface configured to contact tissue;
at least one base;
at least one inflatable cell coupled between said at least one base and said at least one flexible tissue interface;
at least one inflation port in said at least one inflatable cell, wherein said at least one inflatable cell is configured to expand when inflated via said at least one inflation port,
wherein said base comprises at least one connector configured to connect said inflatable actuator unit to at least one additional inflatable actuator unit and to allow movement of an inflatable actuator unit relative to an adjacent inflatable actuator unit.
Example 47. An inflatable actuator unit according to example 46, wherein said at least one connector comprises at least one of, a joint, a hinge and/or a swivel connector.
Example 48. An inflatable actuator unit according to any one of examples 46 or 47, wherein a maximal dimension of said inflatable actuator unit is up to 20 mm.
Example 49. A body implant comprising:
an array of plurality of inflatable actuator units according to example 46 coupled to each other, wherein said array is configured to conform to a curvature of a body tissue by movement of one or more inflatable actuator units relative to other inflatable actuator units in the array.
Example 50. A body implant, comprising:
an array of actuators formed from shape memory alloy, wherein said actuators are interconnected by shape memory alloy bridges;
wherein at least one actuator of said actuators is configured to expand and contract, and to move laterally relative to other actuators in said array when heated.
Example 51. A multi-unit body implant, comprising:
a plurality of single unit implants coupled to each other, wherein each single unit implant comprises:
least one tissue interface configured to be placed in contact with a body tissue;
at least one expandable actuator coupled to said at least one tissue interface;
at least one connector configured to connect each single unit implant to at least one different single unit implant of said plurality of single unit implants, wherein a maximal dimension of each single unit is up to 20 mm.
Example 52. An implant according to example 51, wherein said at least one expandable actuator comprises at least one inflatable chamber, and wherein said at least one expandable actuator is configured to expand when said at least one inflatable chamber is inflated.
Example 53. An implant according to example 52, wherein said at least one expandable actuator is formed from shape memory alloy, and is configured to expand when heated above a predetermined temperature level.
Example 54. An implant according to any one of examples 51 to 53, wherein each single unit implant comprises a base coupled to said at least one expandable actuator opposite to said at least one tissue interface, wherein said base comprises one or more openings which are shaped and sized to allow passage of a screw or a nail through said base and into said tissue to couple said single unit implant to said tissue.
Example 55. A body implant, comprising:
at least one implant cover having a tissue contacting surface and at least one opposite surface, wherein said at least one implant cover comprises at least one central portion, at least one edge portion configured to couple said body implant to tissue, and at least one hinge portion therebetween;
at least one expandable actuator contacting said at least one opposite surface of said at least one central portion, wherein said at least one expandable actuator is configured to move from a collapsed state to an expanded state;
wherein when said at least one expandable actuator expands, said at least one expandable actuator pushes said at least one central portion relative to said at least one edge portion using said hinge portion to acquire a continuous tissue contacting surface of said at least one implant cover.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings and images. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a flow chart of a process for tissue reshaping, according to some exemplary embodiments of the invention;

FIGS. 2A-2B are schematic illustrations showing an energizing effect of actuators of an implant, according to some exemplary embodiments of the invention;

FIGS. 2C-2D are schematic illustrations showing tissue shaping using an implant having at least one solid actuator, for example an actuator, according to some exemplary embodiments of the invention;

FIGS. 3A-3C are block diagrams of implants, according to some exemplary embodiments of the invention;

FIG. 3D is a flow chart of a process for fitting an implant to a particular implantation site and/or for a specific treatment, according to some exemplary embodiments of the invention;

FIG. 4 is a flow chart of a process for face shaping, according to some exemplary embodiments of the invention;

FIGS. 5A-5D are images of actuators shaped as spiral springs, according to some exemplary embodiments of the invention;

FIGS. 5E and 5F are schematic illustrations of an implant comprising a plurality of spiral actuators, according to some exemplary embodiments of the invention;

FIGS. 6A-6D are schematic illustrations of solid actuators having a rectangular base, according to some exemplary embodiments of the invention;

FIGS. 7A-7F are schematic illustrations of an array of spiral actuators according to some exemplary embodiments of the invention;

FIGS. 7G-7J are schematic illustrations of arrays of spiral actuators, according to some exemplary embodiments of the invention;

FIGS. 7K and 7L are schematic illustrations showing shaping of implants, according to some exemplary embodiments of the invention;

FIGS. 8A-8D are schematic illustrations of an implant comprising a plurality of spiral actuators, according to some exemplary embodiments of the invention;

FIG. 8E is a schematic illustration of a spiral actuator and a screw passing through an opening in the spiral actuator, according to some exemplary embodiments of the invention;

FIGS. 9A-9G are schematic illustrations showing spring actuator attachments and covers, according to some exemplary embodiments of the invention;

FIGS. 10A-10C are schematic illustrations of implants having a single actuator, according to some exemplary embodiments of the invention;

FIGS. 11A-11D are schematic illustrations of implants having a plurality of solid implants, according to some exemplary embodiments of the invention;

FIGS. 12A-12B are schematic illustrations of modular implants formed from single unit implants, according to some exemplary embodiments of the invention;

FIGS. 12C-12F are schematic illustrations of an implant within the body, where a tissue contacting surface of an implant cover acquires a uniform smooth shape following expansion of the cover, according to some exemplary embodiments of the invention;

FIGS. 13A-13B are schematic illustrations of wave spring actuators, according to some exemplary embodiments of the invention;

FIGS. 14A-14E are schematic illustrations of enclosed expandable implants, according to some exemplary embodiments of the invention;

FIGS. 15A-15G are schematic illustrations of a flexible implant, according to some exemplary embodiments of the invention;

FIG. 16 is a schematic illustration of a flexible implant located at a chin region of the skull, according to some exemplary embodiments of the invention;

FIGS. 17A-17D are schematic illustrations of an implant comprising a plurality of actuators coupled between two layers of material, for example mesh material, accoridng to some exemplary embodiments of the invention;

FIGS. 18A-18D are schematic illustrations of an implant comprising a plurality of isolated actuators, according to some exemplary embodiments of the invention;

FIGS. 19A-19C are schematic illustrations of an implant comprising a plurality of isolated spring actuators, according to some exemplary embodiments of the invention;

FIGS. 20A-20C are schematic illustrations of an implant comprising a plurality of isolated spring actuators positioned within sockets in a tissue interface, according to some exemplary embodiments of the invention;

FIGS. 21A-21G are schematic illustrations of an implant comprising a plurality of thin expandable actuators, according to some exemplary embodiments of the invention;

FIGS. 22A-22B are schematic illustrations of an implant comprising An inflatable chamber in a collapsed deflated state (22A) and in an expanded inflated state (22B), according to some exemplary embodiments of the invention;

FIGS. 22C-22E are schematic illustrations of an implant comprising a plurality of inflatable actuators, according to some exemplary embodiments of the invention;

FIGS. 23A-23E are schematic illustrations of modular implant comprising a plurality of single unit actuators or implants, according to some exemplary embodiments of the invention;

FIGS. 24A-24C are schematic illustrations of an implant comprising a plurality of inflatable actuators optionally arranged in an array, according to some exemplary embodiments of the invention;

FIGS. 24D-24E are schematic illustrations showing assembly of inflatable actuators to form an implant, according to some exemplary embodiments of the invention;

FIGS. 25A-25C are schematic illustrations showing assembly of an actuators array, according to some exemplary embodiments of the invention;

FIG. 25D is a schematic illustration showing coupling of actuators to a single base, for example via snap connection, according to some exemplary embodiments of the invention;

FIG. 25E is a schematic illustration showing a head to tail orientation arrangement of actuators on a base, according to some exemplary embodiments of the invention;

FIGS. 26A-26E are schematic illustrations showing an implant assembly, according to some exemplary embodiments of the invention;

FIGS. 27A-27C are schematic illustrations showing an implant assembly, according to some additional exemplary embodiments of the invention;

FIGS. 28A-28D are schematic illustrations showing an implant which comprises a chamber and an inner actuators array, according to some exemplary embodiments of the invention; and

FIG. 28E is an image showing a use of the implant shown in FIGS. 28A-28D, as a temple implant, according to some exemplary embodiments of the invention.

### DESCRIPTION OF SPECIFIC EMBODIMENTS OF THE INVENTION

The present invention, in some embodiments thereof, relates to shaping tissue and, more particularly, but not exclusively, to shaping tissue using adjustable implants.

An aspect of some embodiments of the invention relates to shaping body tissue by selectively energizing at least one actuator, for example a solid actuator, within a body implant, for example a filler implant by exposing the at least one actuator to external energy from outside the body. In some embodiments, the implant comprises a plurality of actuators, and at least one actuator of the plurality of actuators is selectively energized. In some embodiments, the energizing comprises delivery of energy to the at least one solid actuator within the implant that will cause the actuator to expand and/or contract. In some embodiments, expansion or collapse of the actuator reshapes, for example locally reshapes, the filler implant leading to change of the tissue shape contacting the implant. In some embodiments, energizing comprises transmitting energy across a skin surface to the at least one actuator, without physically penetrating through the skin. In some embodiments, the energy is applied directly on the solid actuator or is delivered to the solid actuator via tissues contacting the solid actuator. In some embodiments, the energy is delivered in a form of waves through the body to the actuator or to tissue contacting the actuator.

According to some exemplary embodiments, the actuators are apertured actuators, comprising one or more openings. In some embodiments, the one or more openings crosses a body of an actuator. In some embodiments, an actuator is shaped as a spring having apertures crossing through a body of the spring.

According to some embodiments, an energy used to energize the actuator is delivered in the form of waves, for example ultrasonic waves, radiofrequency waves, an electromagnetic field and electromagnetic waves. In some embodiments, the energy is delivered from at least one energy source located outside the body and/or from at least one energy source located within the body, for example within a body cavity or a body lumen. In some embodiments, the term external energy means an energy generated from an energy source located outside the body.

According to some exemplary embodiments, the delivered energy comprises thermal energy delivered by heating, for example induction heating, magnetic inductance, and electromagnetic induction. Alternatively, the energy is delivered using at least one of, ultrasound, radiofrequency, and infrared.

In some embodiments, induction heating parameters include generating magnetic inductance with a frequency in a range of 25 Khz-1000 Khz, for example 25 Khz-200 Khz, 100 Khz-400 Khz, 200 Khz-600 Khz, 500 Khz-1000 Khz, with a frequency of 50 Khz, 100 Khz or any intermediate, smaller or larger frequency or range of frequencies. In some embodiments, the magnetic inductance is generated to induce heating within a time period in a range between 15 sec-120 sec, for example 15 sec-50 sec, 30 sec-100 sec, 70 sec-120 sec, for 40 sec, 60 sec, 100 sec, or any intermediate, smaller or larger value or range of values.

According to some embodiments, the actuators are arranged in an array, for example a two dimensional array, a linear array, or a round array, within the implant. In some embodiments, the implant is modular, and the actuators are configured to be assembled or disassembled from the array, for example to conform to size and/or shape of an implantation site. In some embodiments, a modular implant comprises a plurality of similar and/or different units, assembled to form a single implant. In some embodiments, each unit is at least one uncovered actuator, for example an aperture or an inflatable actuator, at least one covered or enclosed actuator, a unit that includes an actuator an a base, or a unit that includes an actuator coupled to a base and enclosed at least partly within a cover. In some embodiments, a modular implant is formed by aligning two or more units side by side, or one above the other. Additionally, in a modular implant two or more units are coupled to a shared element of the implant, for example to a shared base, a shared cover, a shared inflation tube. In some embodiments, in a modular implant, the two or more units can be reversible assembled to form the modular implant, for example can be reversibly coupled in a way that allows decoupling without irreversibly deforming the implant or the units.

According to some exemplary embodiments, two or more units are connected to each other, for example side by side, via at least one connector of one or both of the units. In some embodiments, the connector is configured to allow movement of each unit relative to an adjacent interconnected unit. In some embodiments, the connector comprises at least one of, a joint, a hinge and/or a swivel connector.

According to some embodiments, a maximal dimension of each unit, for example a maximal width, a maximal thickness and/or a maximal length is up to 20 mm, for example up to 15 mm, up to 10 mm, up to 7 mm, up to 5 mm, up to 3 mm, up to 2 mm, or any intermediate, smaller or larger value. In some embodiments, the maximal dimension is dimension of the unit when the unit is at compressed state, for example a maximal compact state.

In some embodiments, the actuators are spaced-apart from other actuators within the array. In some embodiments, each of the actuators or at least some of the actuators within an array are configured to expand and/or contract independently from other actuators.

An aspect of some embodiments relates to an inflatable actuator which comprises at least one inflatable cell configured to expand when inflated. In some embodiments the at least one inflatable cell is positioned between at least one flexible tissue interface configured to contact soft tissue, and at least one base. In some embodiments, the base of the inflatable actuator is configured to be coupled to bone tissue, for example using at least one screw, pin or nail.

According to some exemplary embodiments, the inflatable actuator comprises at least one inflation port in said at least one inflatable cell. In some embodiments, inflation of said at least one inflatable cell via said at least one inflation port expands the at least one inflatable cell increases a distance between the at least one flexible tissue interface and the base.

According to some exemplary embodiments, the at least one inflation port is fluidically coupled to at least one channel, for example a fluid channel that passes through the at least one flexible interface. Alternatively or additionally, the at least one channel passes through the base of the actuator.

According to some exemplary embodiments, a body implant comprises a plurality of inflatable actuators, coupled to each other using at least one connector or at least one fastener. In some embodiments, the at least one connector comprises a hinge configured to allow movement of at least one inflatable actuator relative to a different actuator of the implant, for example relative to an adjacent actuator.

An aspect of some embodiments relates to a body implant comprising a base and a plurality of inflatable actuators coupled to the base. In some embodiments, the base comprises at least one inflation channel fluidically coupled to at least one inflatable cell of each of the inflatable actuators. In some embodiments, the at least one inflation channel is configured to allow fluid flow into the inflatable cells, for example to allow inflation and expansion of the inflatable actuators. Alternatively, each inflatable cell comprises a separate inflation port, for example to allow inflation of each inflatable cell independently from the rest of the inflatable cells of the implant.

According to some exemplary embodiments, each of the inflatable actuators comprises at least one fastener, for example to allow fastening of the inflatable actuator to the implant base. In some embodiments, the at least one fastener comprises an interference lock, or a snap-fit fastener. According to some exemplary embodiments, each of the inflatable actuators comprises a plug-in portion coupled to the inflatable cell, configured to allow easy coupling between the inflatable cell and the at least one inflation channel in the implant base. In some embodiments, the plug-in portion comprises a syringe.

An aspect of some embodiments relates to an array of expandable compartments, each having at least one solid actuator. In some embodiments, each of the expandable compartments moves between a collapsed state and an expanded state when the at least one solid actuator within an expandable state is energized. In some embodiments, the array comprises a two-dimensional (2D array) or a linear array of expandable compartments. In some embodiments, the array comprises a base layer, for example a flexible base layer, to which the expandable compartments or the at least one solid actuator is coupled.

According to some embodiments, the expandable compartments are configured to expand and contract along a similar axis. Alternatively, at least some of the expandable compartments are configured to expand in a different axis relative to other expandable compartments of the array.

According to some embodiments, the array is a modular array, where one or more expandable compartments can be reversibly assembled to form the array and/or can be disassembled from an array. In some embodiments, the expandable compartments of an array are movable relative to each other, for example at least one expandable compartment is configured to laterally move relative to at least one different expandable compartment of the same array.

An aspect of some embodiments relates to a modular implant, for example a tissue shaping adjustable implant comprising a plurality of single units of expandable implants. In some embodiments, each of the single unit implants comprises at least one expandable actuator, and at least one tissue interface coupled to the at least one expandable actuator. In some embodiments, the plurality of single unit implants are coupled to each other using at least one connector, for example a hinge. In some embodiments, the plurality of single unit implants are reversibly coupled to each other, for example to allow disassembly of the modular implant. Alternatively, the plurality of single unit implants are irreversibly coupled to each other.

According to some exemplary embodiments, actuators of the single unit implants comprise solid actuators, for example actuators formed from shape memory alloy. Alternatively or additionally, actuators of the single unit implants comprise inflatable cells, for example balloons.

An aspect of some embodiments relates to a body implant comprising an array of actuators formed from shape memory alloy. In some embodiments, the actuators are interconnected by shape memory alloy bridges that are configured to allow lateral movement of actuators relative to other actuators within the array, when heated, for example to a temperature that is higher than a predetermined temperature level. In some embodiments, heating of the actuators array expands the actuators in an axis that is perpendicular to a long axis of the array.

An aspect of some embodiments relates to a body implant comprising at least one implant cover comprising a central portion and an edge portion interconnected by a hinge portion, for example a hinge portion, of the cover. In some embodiments, when the body implant is in a collapsed state, a tissue contacting surface of the cover is uneven and comprises at least one indentation, for example in the hinge portion. In some embodiments, when the implant expands, the central portion moves relative to the edge portion to generate an even, continuous and/or smooth tissue contacting surface.

According to some exemplary embodiments, the cover surrounds at least one expandable actuator positioned within the implant, and is optionally in contact with the central portion of the cover. In some embodiments, expansion of the at least one actuator expands the implant and moves the central portion relative to the edge portion of the cover. In some embodiments, the edge portion of the cover is used to couple, for example anchor, the implant to tissue, for example to bone tissue. In some embodiments, the edge portion inwardly bends towards the implant inner lumen defined by the cover, for example to allow coupling of the implant to bone tissue via one or more openings in the edge portion.

In some embodiments, a thickness of a body implant in a compact state, for example a collapsed state is a value in a range between 0.5 mm to 4 mm, for example in a range between 0.5 mm to 2 mm, 1 mm to 3 mm, 1 mm to 4 mm, 2 mm to 4 mm or any intermediate, smaller or larger value or range of values. In some embodiments, a thickness of a body implant in a fully expanded state is a value within a range between 3 mm and 25 mm, for example between 3 mm and 7 mm, between 5 mm and 10 mm, between 7 mm and 17 mm, between 12 mm and 25 mm, or any intermediate, smaller or larger value or range of values. In some embodiments, a maximal width and/or a maximal length of the body implant is a value within a range between 10 mm to 80 mm, for example between 10 mm to 30 mm, between 10 mm to 20 mm, between 15 mm to 40 mm, between 20 mm to 70 mm, between 40 mm to 100 mm, or any intermediate, smaller or larger value or range of values. In some embodiments, a weight of the body implant is a value in a range between 0.1 gr and 50 gr, for example between 10 gr and 30 gr, between 15 gr and 40 gr, between 20 gr and 50 gr, or any intermediate value or range of values.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

### Exemplary General Tissue Shaping Process

According to some exemplary embodiments, tissue shaping is performed for aesthetic and/or functional reasons. In some embodiments, tissue shaping is performed, for example in a face, in order to correct birth defects, defects caused by surgical removal of tissue, for example when a tumor is removed, a defect caused by trauma to the tissue, for example an accident leading to changes in bone structure and/or changes in soft tissue. In some embodiments, tissue shaping is performed in order to correct aesthetic defects, for example age-related aesthetic defects or aesthetic defects that cause interference to the patient. In some embodiments, the tissue shaping is performed in order to correct age-related changes, for example age-related changes of the face or any body part.

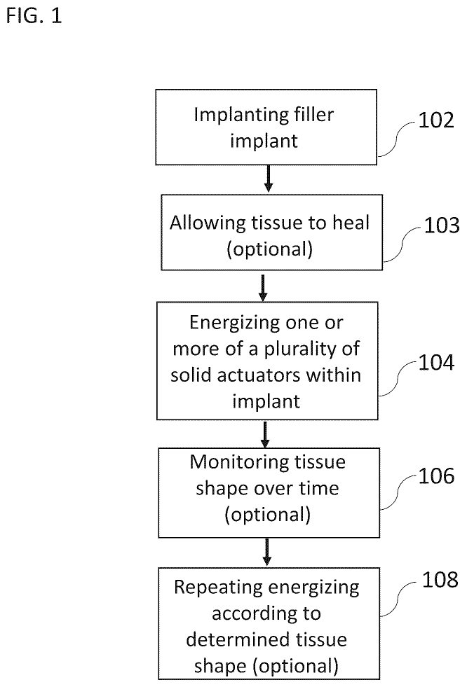

Reference is now made to FIG. 1 depicting a general process for tissue shaping, according to some exemplary embodiments of the invention.

According to some exemplary embodiments, an implant, for example a filler implant, is implanted in a body, at block 102. In some embodiments, the implant comprises a plurality of actuators. In some embodiments, the plurality of actuators are arranged in an array, and are optionally spaced apart from each other. In some embodiments, each or at least one of the actuators is configured to expand and/or to contract in at least one axis. In some embodiments, each or at least one of the actuators is configured to expand and/or to contract in a similar extent in all directions. Alternatively, each or at least one of the actuators is configured to extend and/or to contract in a larger extent in at least one first direction relative to at least one second direction.

According to some exemplary embodiments, the tissue is allowed to heal, at block 103. In some embodiments, the tissue is allowed to heal after implantation of the implant at block 102, and prior to energizing at least one implant actuator at block 104. Alternatively, at least one implant actuator is energized during an implantation surgery and/or when the patient is still in a surgical operating room, or in a clinic following implantation at block 102.

According to some exemplary embodiments, one or more of the actuators within the implant is selectively energized, at block 104. In some embodiments, one or more of the actuators is energized by delivery of energy through a skin surface. In some embodiments, energizing of the one or more actuators comprises heating the one or more actuators. Alternatively or additionally, energizing comprises delivering energy, for example in the form of ultrasound waves or a magnetic field, to the one or more actuators. In some embodiments, the energy is delivered from outside the body, and optionally, without physically penetrating through skin surface in order to reach the one or more actuators. In some embodiments, the energy is delivered using at least one of, ultrasound, radiofrequency (RF), and light for example infra-red or laser. In some embodiments, energizing comprising heating the one or more of the solid actuators by induction heating, for example by exposing the one or more solid actuators to a magnetic field. In some embodiments, the magnetic field is an alternating magnetic field, optionally generated outside the body. Optionally, the energy is delivered from within the body, for example by placing an energy generating device within a body cavity.

According to some exemplary embodiments, energizing the one or more of the actuators leads to expansion or contraction of the one or more actuators resulting with localized shaping of the tissue.

According to some exemplary embodiments, a shape of the tissue over time is optionally monitored, at block 106. In some embodiments, the shape of the tissue is monitored over time, for example to identify changes in the tissue shape, for example changed caused by at least one of, healing of the body, bone growth, bone movement, soft tissue growth and/or loss of tissue.

According to some exemplary embodiments, energizing of the one or more actuators of the implant is repeated at block 108. In some embodiments, the energizing is repeated, for example to reshape the tissue according to the changes in tissue identified at block 108. In some embodiments, the one or more actuators are energized, for example to cause the one or more actuators to expand or collapse, or to increase or reduced previous expansion or previous collapse of the one or more actuators.

### Exemplary Tissue Shaping

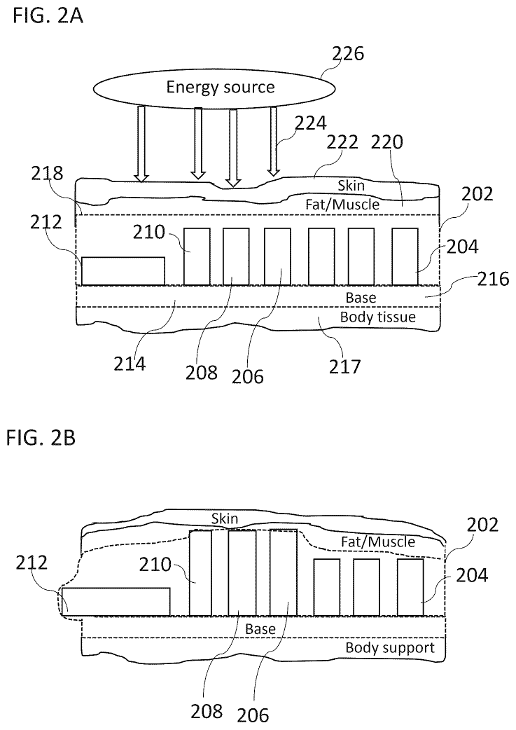

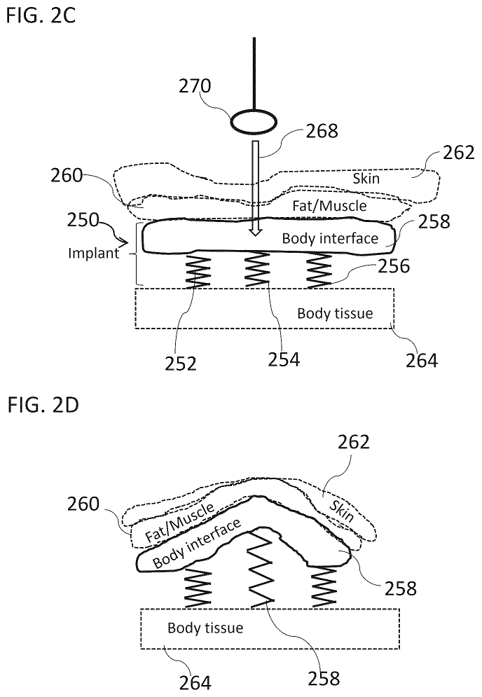

Reference is now made to FIGS. 2A-2B, depicting an effect of energizing on implant actuators action, according to some exemplary embodiments of the invention.

According to some exemplary embodiments, an implant 202 comprises a plurality of actuators, for example actuators 204, 206 and 208, that are configured to expand or to contract in response to energy delivered to the actuators. In some embodiments, the actuators are solid actuators, for example in a form of a spring shaped as a spiral or a wave spring. In some embodiments, the actuators are spaced-apart from each other, and are configured to independently expand or contract when exposed to energy. In some embodiments, the actuators are aligned within the implant to expand and contract in a same direction, for example along the same axis. Alternatively, at least some of the actuators within an implant are oriented in a way that allows expansion and contraction at different directions compared to other actuators of the implant.

According to some exemplary embodiments, the actuators comprise a shape memory alloy, for example Nitinol that is configured to change shape when heated.

According to some exemplary embodiments, for example as shown in FIGS. 2A and 2B, an implant 202 comprises a plurality of actuators, for example actuators 204, 206, 208, 210 and 212. In some embodiments, at least some of the actuators are arranged and aligned along a similar axis, for example actuators 204, 206, 208 and 210. In some embodiments, at least some of the actuators, for example actuator 212 is oriented at a different direction relative to other actuator of the implant. In some embodiments, the actuators are coupled to a material layer, for example a base layer 216 or a tissue interface layer 218. In some embodiments, the material layer is flexible, for example to allow conformation of the implant to body tissue shape and/or anatomy. In some embodiments, for example as shown in FIGS. 2A and 2B, the implant 202 is implanted underneath a fat layer or a muscle layer 220 located under the skin 222. Alternatively, the implant is implanted directly under the skin. In some embodiments, a base layer 216 of the implant is placed in contact with body tissue 217, for example with bone tissue.

According to some exemplary embodiments, energy 224 delivered from an energy source 226 located outside the body towards the implant 202 selectively energizes one or more of the actuators within the implant, for example as shown in FIG. 2A. In some embodiments, the energy 224 selectively energizes actuators 206, 208, 210 and 212, without affecting or with minimal effect on other actuators of the implant, for example actuator 204. In some embodiments, the energy 224 selectively heats one or more of the actuators, for example actuators 206, 208, 210 and 212.

According to some exemplary embodiments, for example as shown in FIG. 2B, selectively energizing, for example selectively heating of the actuators leads to expansion of the actuators 206, 208, 210 and 212, within the implant that pushes the surrounding tissue contacting the implant, for example fat and/or muscle tissue 220 and/or the skin tissue 220 causing reshaping of the outer body surface, for example changing curvature of the outer surface of the skin above the implant or above the energized actuator.

Reference is now made to FIGS. 2C and 2D depicting tissue shaping using an implant comprising at least one solid actuator, according to some exemplary embodiments of the invention.

According to some exemplary embodiments, implant 250 comprises one or more solid actuators shaped as springs, for example actuators 252, 254 and 256. In some embodiments, the springs are configured to move between an expanded state and a compressed state. In some embodiments, the springs are configured to expand in a relaxed state.

According to some exemplary embodiments, the springs 252, 254 and 256 are coupled to a body interface 258, for example a soft body interface. In some embodiments, for example as shown in FIG. 2C, the implant 250 is implanted underneath the skin 262, and optionally underneath fat and/or muscle tissue 260. In some embodiments, the implant 250 is oriented to place the springs in contact with body tissue 264, for example bone, and the body interface 258 is in contact with the skin 262, muscle and/or fat tissue 260.

According to some exemplary embodiments, delivery of energy 268 to the springs, for example from energy source 270 leads to expansion of the springs. In some embodiments, the energy source 270 comprises a source of a magnetic field, for example an alternating magnetic field. In some embodiments, the energy 268 comprises a magnetic field. In some embodiments, the springs comprise an electrically conductive material that when exposed to the magnetic field are heated. In some embodiments, induction of heating relaxes the springs and moves the heated springs into an expanded state, for example as shown in FIG. 2D.

According to some exemplary embodiments, selective exposure of one or more of the springs to energy 268, for example spring 258 leads to expansion of the spring 258. In some embodiments, the expansion of a selected one or more springs is a relative expansion compared to expansion of other springs of an implant.

According to some exemplary embodiments, relative expansion of one or more springs compared to other springs of an implant is achieved by using springs that have different characteristics compared to other springs. For example, one or more of the springs can be formed with at least one of, a different cross-sectional thickness, a different radius, and/or different number of spiral turns, etc. Thus, by applying the same energy 268 to the springs, their respective different characteristics provide a different expansion relative to other springs. In some embodiments, different expansion in different springs can be facilitated by localizing the delivery of different amounts of energy to each of the springs.

According to some exemplary embodiments, for example as shown in FIG. 2D, expansion of one or more of the springs pushes the body interface 258 against at least one of the skin 262, fat and/or muscle layers of the body, leading to change in a shape of the tissue, for example a change in a curvature of an external surface of the tissue.

In some embodiments, expansion of one or more of the springs between a rigid body tissue, for example, and a soft body tissue, for example skin, fat and muscle tissue, results with outwardly pushing of the soft tissue away from the rigid body tissue.

### Exemplary Implant

According to some exemplary embodiments, an implant is used in shaping a shape of a tissue, for example facial tissue or any other tissue of the body. In some embodiments, tissue shaping comprises changing an external curvature or external contour of the tissue. Alternatively, or additionally, the implant is used to fill volumes of missing tissue in the body, formed by at least one of, trauma to the body, removal of tumorigenic tissue, and removal of inflammatory tissue. In some embodiments, removal of tissue results with a change of a body contour due to collapse of the skin surface into the formed void. In some embodiments, the implant is used to fill the formed void and by pressing outwardly against the skin to reshape the outer surface of the body distally to the filled void. Optionally, the implant is used to restore the shape of the body outer surface to a shape of the body outer surface prior to the void formation.

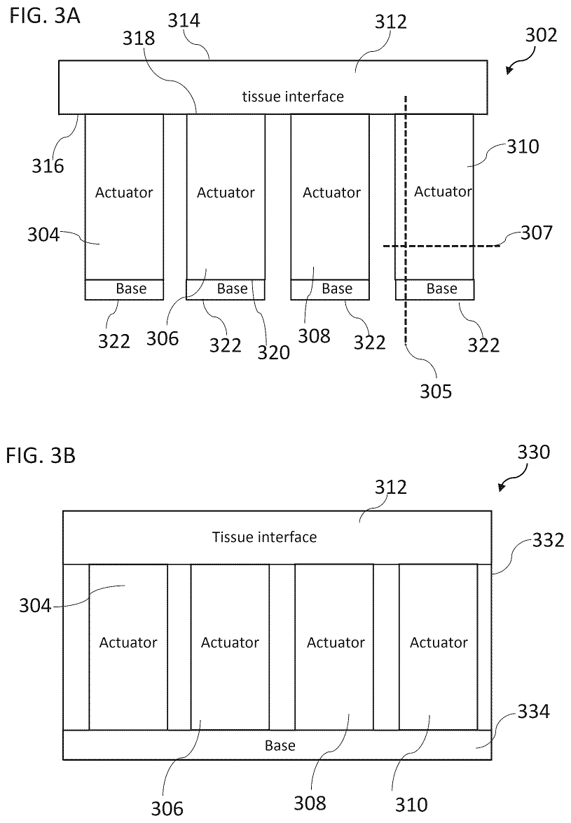

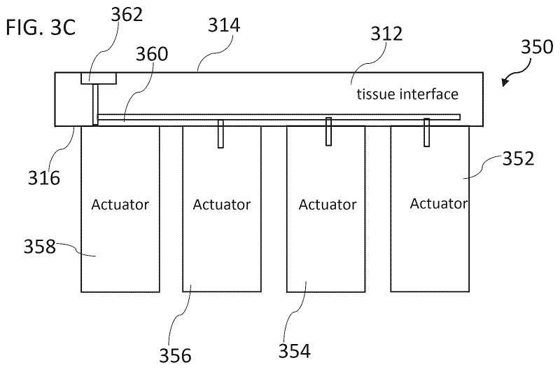

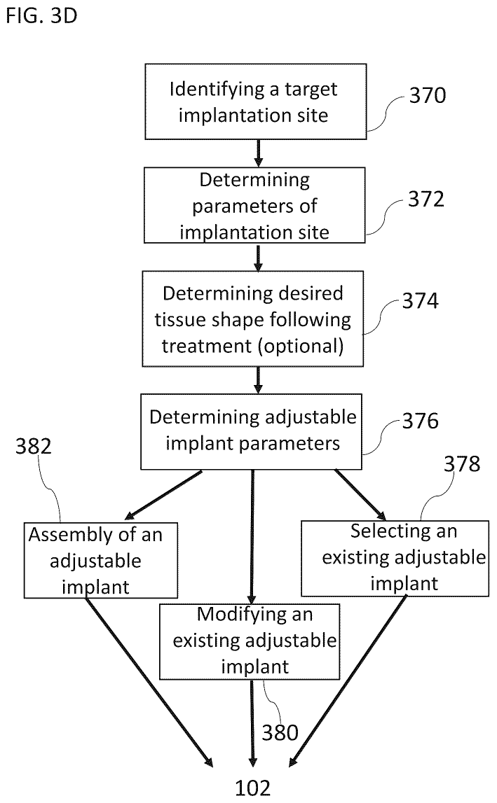

Reference is now made to FIGS. 3A and 3B, depicting implants, according to some exemplary embodiments of the invention.

According to some exemplary embodiments, for example as shown in FIG. 3A, an implant 302 comprises at least one actuator or two or more actuators, for example actuators 304, 306, 308 and 310. In some embodiments, each of the actuators is configured to move between a compressed state and an expanded state. In some embodiments, each of the actuators or at least some of the actuators are configured to expand or compress in a selected axis, for example in a long axis 305 of an actuator. In some embodiments, expansion or compression of each actuator or at least some of the actuators of an implant is larger in a specific axis, for example in the long axis 305, compared to expansion or compression in a different axis, for example short axis 307 of the actuator.

According to some exemplary embodiments, at least some or all of the actuators are solid actuators. In some embodiments, at least some or all of the actuators comprise springs or are shaped as a spring. In some embodiments, the actuators, for example the springs are formed from a shape memory material (SMM), comprising shape memory alloy (SMA) and/or shape memory polymer (SMP), for example as described in Huang et. al., (2010). In some embodiments, the actuators, for example the springs are configured to recover their original shape from a significant and seemingly plastic deformation when a particular stimulus is applied.

According to some exemplary embodiments, when the actuators are exposed to a stimulus, for example in the form of an energy, the actuators expand, optionally to their original shape. Alternatively, when the actuators are exposed to a stimulus, for example in the form of an energy, the actuators collapse.

According to some exemplary embodiments, at least some or all of the actuators 304, 306, 308 and 310 are formed from a shape memory alloy, for example copper-aluminum-nickel alloy, a nickel-titanium (NiTi) alloy (Nitinol). In some embodiments, when the actuators formed from a shape-memory alloy are heated, they expand, optionally acquiring an original shape. Alternatively or additionally, the at least some or all of the actuators 304, 306, 308 and 310 are formed from a different shape memory material, for example a shape memory polymer According to some exemplary embodiments, the each of the actuators is positioned within a separate isolating sleeve, configured to isolate the actuator, for example a spring, from the surrounding environment. In some embodiments, the isolating sleeve is configured to thermally isolate the actuator from the surrounding environment, for example from tissue surrounding the implant following implantation of the implant in the body.

According to some exemplary embodiments, the implant 302 comprises a tissue interface 312 configured to be placed in contact with a tissue of the body, for example with soft tissue of the body. In some embodiments, the soft tissue of the body comprises at least one of skin, muscle and fat tissue. In some embodiments, the tissue interface 312 comprises a pad or a cushion. In some embodiments, the tissue interface 312 is soft and/or flexible, for example to conform to the shape of the body soft tissue. Optionally, the tissue interface 312 is filled with a fluid or a viscous material, for example with gel.

According to some exemplary embodiments, the tissue interface 312, for example a first tissue interface, comprises at least one first surface 314 configured to contact the body tissue, for example soft tissue of the body, and at least one second surface 316. Optionally, the at least one second surface is opposite to the at least one first surface 314. In some embodiments, the actuators are coupled to the at least one second surface 316. In some embodiments, applying a force towards the at least one second surface, for example when one or more of the actuators expand, pushes the at least one surface 314 against the tissue. In some embodiments, the applied force changes a shape of the tissue interface 312, for example the shape or curvature of the at least one surface 314 in a level that is relative to the force applied on the at least one second surface 318. Optionally, local application of force on the at least one second surface 316 locally deforms the shape of a region of the tissue interface 312 which is aligned with a direction of the applied force.

According to some exemplary embodiments, each of the actuators, for example springs, comprise a first end 318 and a second opposite end 320. In some embodiments, the actuators are coupled to the at least one second surface 316 of the tissue interface 312 by the first end 318 of each actuator.

According to some exemplary embodiments, the actuators comprise an inflatable chamber, for example a balloon. In some embodiments, inflation of the inflatable chamber expands the chamber and applies force against the surface 316 leading to upwardly bending of the tissue interface 312.

According to some exemplary embodiments, the implant 302 comprises a separate base 322 for each of the actuators, coupled to the at least one second end 320 of each actuator. In some embodiments, the base 322 is configured to allow a stable contact between the actuator and a tissue of the body, for example a rigid tissue of the body. In some embodiments, the rigid tissue of the body comprises a bone tissue. In some embodiments, the base 322 is configured to anchor an actuator to the rigid body tissue, for example via openings in the base and/or in one or more of the actuators that are suitable for receiving a nail or a screw. Alternatively or additionally, a cover, for example the tissue interface 312 extends to the bone tissue, and is fixed to the bone tissue using nails or screws that penetrate through the cover into the bone. Alternatively, the implant, for example at least one of a base, an actuator or the cover is attached to the bone tissue using adhesive, for example glue.

According to some exemplary embodiments, the base 322 is stiffer than the tissue interface 312. In some embodiments, coupling the actuators to the tissue, anchors the implant 302 within the body.

According to some exemplary embodiments, the actuators are spaced-apart from each other, for example to allow tissue ingrowth into the implant in regions located between adjacent actuators.

According to some exemplary embodiments, for example as shown in FIG. 3B, an implant 330 comprises a hollow body 332 with at least one actuator or a plurality of actuators, for example actuators 304, 306, 308 and 310 positioned within the body 322. In some embodiments, the actuators are positioned between the tissue interface 312 and a shared base 334 configured to contact rigid tissue of the body, for example bone. In some embodiments, the base 334 is configured to anchor the implant 330 to the body, for example to a rigid tissue of the body.

According to some exemplary embodiments, the hollow body 332 is an enclosed hollow body configured to isolate the actuators from the surrounding tissue when implant 330 is implanted inside the body. Alternatively, the hollow body 332 is at least partly perforated, for example the base 334 is perforated, in order to allow tissue ingrowth into the implant.

According to some exemplary embodiments, at least one or all of the actuators are substantially perpendicular to the tissue interface. Alternatively, at least one or all of the actuators are positioned at an angle selected from a range between 30 degrees and 90 degrees relative to the tissue interface 312, for example at an angle selected from a range between 40 degrees and 90 degrees, at an angle selected from a range between 40 degrees and 50 degrees, or any intermediate, smaller or larger range of angles.

Reference is now made to FIG. 3C, depicting an implant having a tissue interface coupled to two or more inflatable chambers, according to some exemplary embodiments of the invention. According to some exemplary embodiments, an implant, for example implant 350 comprises a tissue interface 312 and two or more inflatable actuators, for example actuators 352, 354, 356 and 358 coupled to a surface 316 which is opposite to the skin contacting surface 314, for example as described in FIG. 3A. In some embodiments, at least one or all of the inflatable actuators comprise one or more inflatable chambers, configured to inflate by introducing fluid into the inflatable chambers, and optionally to deflate, for example when removing fluid from the inflatable chambers.

According to some exemplary embodiments, fluid is introduced into the inflatable actuators via a fluid flow path, for example channel 360. In some embodiments, the channel interconnects all or at least one of the inflatable actuators to at least one opening 362, optionally comprising a valve, for example a duckbill valve. In some embodiments, the at least one opening 362 is located in the tissue interface, for example in a tissue contacting surface of the tissue interface, for example to allow easy access to the channel 360 from outside the body. In some embodiments, fluid, for example gas or liquid, is introduced and/or is removed from the inflatable actuators using a needle penetrating into the opening 362. Optionally, each of the inflatable actuators is connected to a separate channel and a separate opening, for example to allow selective introduction or removal of fluid from each of the inflatable actuators.

According to some exemplary embodiments, an implant comprises only solid actuators, for example springs, or only inflatable actuators. Alternatively, an implant comprises a combination of solid actuators and inflatable actuators.

According to some exemplary embodiments, an implant is oriented such that the tissue interface contacts tissue underneath the skin and the actuators contact directly or indirectly rigid tissue, for example bone. Alternatively, an implant is oriented such that the tissue interface contacts the bone while a at least one or two or more actuators coupled to the tissue interface push tissue underneath the skin.

### Exemplary Fitting an Implant to a Target Implantation Site

According to some exemplary embodiments, prior to implantation of an adjustable implant there is a need to fit an implant with specific characteristics to a particular implantation site, while optionally taking into consideration, location of the implantation site, shape and/or size of the implantation site, and/or a target shape of the soft tissue following implantation and recovery. In some embodiments, in order to fit an implant for a specific implantation site, an expert can select an implant from a variety of preformed implants. Alternatively, the expert can modify an existing preformed implant. Alternatively, the expert can form a new implant to match the implantation site and/or a future target shape of the tissue.

Reference is now made to FIG. 3D, depicting a process for fitting an implant according to at least one of, size and/or shape of an implantation site, a target shape of a body tissue following implantation, and/or according to a capacity of the implant to be adjusted during recovery to reach the target tissue shape, according to some exemplary embodiments of the invention.

According to some exemplary embodiments, a target implantation site is identified at block 370. In some embodiments, the target implantation site is a site in a head, a limb, a torso and/or a back or is a site at any other location in a subject body. In some embodiments, the target implantation site is identified during or following a surgery, for example a tumor or a mass removal surgery.

According to some exemplary embodiments, one or more parameters of the target implantation site are determined at block 372. In some embodiments, the one or more parameters comprise size, shape, volume, composition of surrounding tissue, and/or proximity to different tissue. In some embodiments, the different tissue comprises at least one of, nerve tissue, blood vessels, bone tissue and/or skin tissue.

According to some exemplary embodiments, a desired tissue shape following implantation, is optionally determined at block 374. In some embodiments, a desired tissue shape following recovery from the implantation procedure is determined at block 374. In some embodiments, the desired tissue shape comprises outer body curvature at the implantation site and/or volume of the tissue.

According to some exemplary embodiments, one or more parameters of the adjustable implant are determined at block 376. In some embodiments, the one or more parameters are determined based on the one or more parameters of the implantation site and/or based on the desired shape following treatment. In some embodiments, the one or more implant parameters comprise at least one of, shape, size, volume, expansion capacity, type of material forming the implant, implant porosity degree, type of actuators, shape of actuators, type of spiral shape of actuators, distribution of actuators in the implant, and/or number of actuators.

According to some exemplary embodiments, an existing implant, for example an already preformed implant, is selected at block 378. In some embodiments, the existing implant is selected out of at least two existing implants, each having one or more different implant parameters. In some embodiments, the existing implant is selected according to the determined adjustable implant parameters, determined at block 376. Optionally, the existing implant is an off the shelf product.

According to some exemplary embodiments, alternatively, an existing adjustable implant is modified, at block 380. In some embodiments, the existing implant is modified according to the determined implant parameters, determined at block 376. In some embodiments, the existing implant is modified, for example by cutting the implant according to a specific shape.

According to some exemplary embodiments, alternatively, a new implant is assembled, at block 382. In some embodiments, the implant is assembled according to the implant parameters determined at block 376. In some embodiments, the implant is assembled at the surgery room, for example prior or during an implantation procedure. Alternatively, the implant is assembled outside the surgery room, prior to the implantation procedure.

According to some exemplary embodiments, after having an implant with the target parameters, or properties, the implant is implanted at the implantation site, for example as described at block 102. Optionally, one or more of the actuators are energized, for example selectively energized prior to implantation. In some embodiments, one or more of the actuators are energized, by heating of the actuators, for example by injection of hot liquid into the implant and/or by exposing the one or more actuators to energy., prior to implantation.

### Exemplary Face and Body Remodeling

According to some exemplary embodiments, the implant is used for shaping the skin for aesthetic reasons, and to treat defects, for example defects caused by trauma to the body and birth defects. In some embodiments, the implant is impanated underneath the skin, and optionally underneath at least one additional tissue layer of the body, for example muscle and fat tissue layers. In some embodiments, the implant is configured to be implanted in one or more regions of the body, for example in a face region, in a head region, a neck region, a region of one or more limbs, an abdomen region, a chest region, a back region, or any region of the body. In some embodiments, the implant is used for shaping of the body region, for example the body region into which it is implanted.

According to some exemplary embodiments, the implant is controllably and/or selectively expanded to push the skin at selected regions, for example to shape the skin surface and/or skin contour into a desired shape. Additionally, or optionally, the implant is used to fill voids or lumens in the body, caused by trauma, a surgical procedure or voids or lumens that are birth defects.

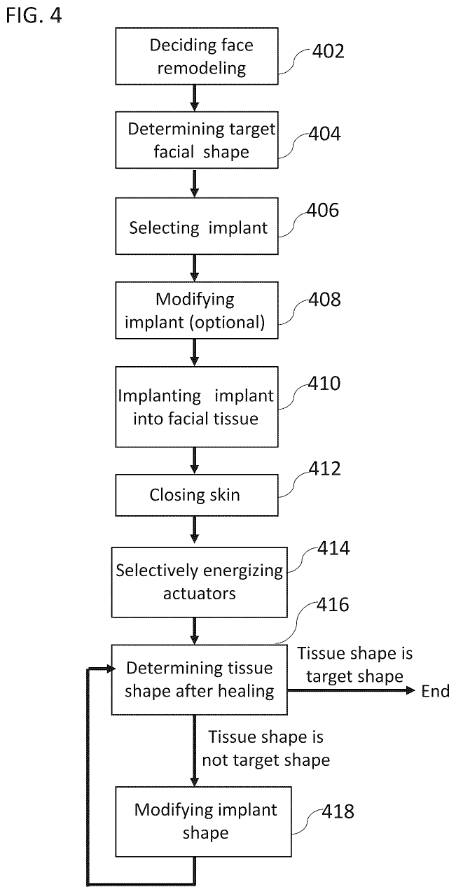

Reference is now made to FIG. 4, depicting a process for face shaping, for example face remodeling, according to some exemplary embodiments of the invention.

According to some exemplary embodiments, face remodeling is decided at block 402. In some embodiments, a face remodeling is decided in order to reach a desired face shape. In some embodiments, deciding to undergo face remodeling comprises determining a part of the face to remodel, for example chin, forehead, cheek.

According to some exemplary embodiments, a target facial shape is determined, at block 404. In some embodiments, determining a target shape comprising determining a shape of a part of the face selected for remodeling after healing from the face remodeling procedure.

According to some exemplary embodiments, an implant is selected at block 406. In some embodiments, the implant is selected according to the determined target facial shape and/or according to a current shape of the facial part selected for remodeling. In some embodiments, an implant with a desired shape, size and/or functionality is selected at block 406.

According to some exemplary embodiments, optionally, the implant is modified at block 408. In some embodiments, at least one of, the shape, structure and/or composition of the implant is optionally modified at block 408 to match the current shape of the facial part selected for remodeling and/or according to the determined target facial shape. In some embodiments, the implant is a modular implant and can optionally modified at block 408, for example by removing or adding actuators to the implant.

According to some exemplary embodiments, the implant is implanted into facial tissue, at block 410. In some embodiments, the implant comprises only two or more actuators coupled to each other that are implanted into the body. Optionally, each of the two or more actuators comprise a separate tissue interface that is configured to push skin. Alternatively, for example as shown in FIGS. 3A-3C, the actuators are coupled to at least one tissue interface, for example a single shared interface.

According to some exemplary embodiments, during implantation of the implant at block 410 the implant and/or the actuators or at least one of the actuators is coupled, for example anchored to bone tissue. Optionally, anchoring of the implant or at least one actuator to bone tissue fixes the implant position within the body and optionally prevents migration of the implant from the implantation site.

According to some exemplary embodiments, the skin is closed at block 412 following the implantation of the implant. In some embodiments, the skin is closed while covering the implant. According to some exemplary embodiments, one or more of the actuators of the implant in energized at block 414. In some embodiments, energizing at least one actuator expands the actuator, and optionally pushes the skin surface. In some embodiments, one or more of the actuators is energized when the patient is still in surgery, and optionally prior to closing the skin at block 412. In some embodiments, one or more of the actuators is selectively energized following closure of the skin while the patient is still undergoing the implantation procedure. Alternatively, the one or more of the actuators is selectively energized after a healing period following the implantation surgery, for example after a healing period of at least 12 hours, of at least 24 hours, of at least 48 hours, of at least 72 hours, after a time period of at least 1 week, after a time period of at least 2 or 3 weeks, after a time period of at least 1 month, or after any intermediate, shorter or longer time period.

According to some exemplary embodiments, selectively energizing one or more of the actuators at block 414 comprises heating the actuators using at least one of, induction heating, ultrasound waves penetrating through the skin towards the implant, radiofrequency (RF) and by injecting warm fluid into the implant. Alternatively, when the actuators comprise at least one inflatable chamber, selectively energizing the actuators comprise inflating the at least one inflatable chamber, for example by introducing fluid into the at least one inflatable chamber.

According to some exemplary embodiments, tissue shape is determined following the selectively energizing and optionally following a healing period, at block 416. In some embodiments, a relation between the tissue shape following the selectively energizing and optionally following a healing period, and a target shape, for example the target facial shape, is determined at block 416. Optionally, the tissue shape following the selectively energizing and optionally following a healing period, is compared to the target facial shape, at block 416. In some embodiments, if the tissue shape is a target shape, then the face remodeling process ends.

According to some exemplary embodiments, if the tissue shape is not a target shape, then the implant shape is modified, at block 418. In some embodiments, the implant shape is modified by energizing at least one of the actuators, and optionally a different actuator from the one energized at block 414. Alternatively or additionally, the implant shape is modified, by applying force on the implant, for example by pressing the implant. In some embodiments, force is applied on the implant during or following the selectively energizing of one or more of the actuators.

According to some exemplary embodiments, when the actuators comprise at least one inflatable chamber, the implant shape is modified, for example by deflating the at least one inflatable chamber and/or by inflating at least one inflatable chamber.

### Exemplary Spiral Springs and Implant

According to some exemplary embodiments, an implant comprises one or more solid actuators, for example a plurality of solid actuators. In some embodiments, the actuators comprise springs that can move between a contracted state, for example a collapsed state and an expanded state when exposed to energy. In some embodiments, the energy heats the springs and allows them to move to a relaxed exposed state. In some embodiments, each spring is formed from a shape-memory alloy, for example Nitinol. In some embodiments, heating of the spring returns the spring shape to a pre-deformed expanded shape.

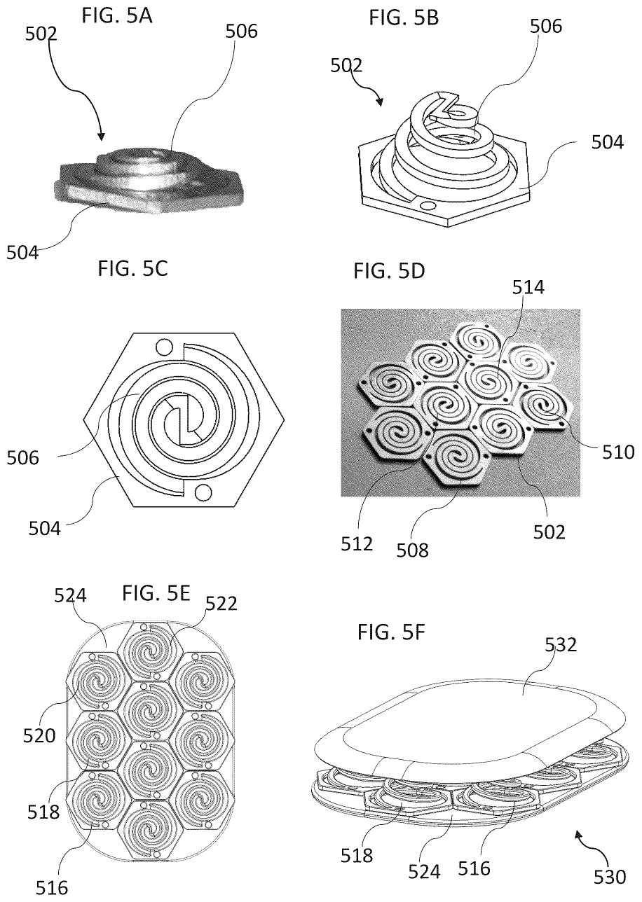

Reference is now made to FIGS. 5A-5D depicting actuators of an implant, where the actuators are shaped as spiral springs, according to some exemplary embodiments of the invention.

According to some exemplary embodiments, for example as shown in FIGS. 5A-5C, a solid actuator comprises at least one spring, for example spiral spring 502. In some embodiments, the spiral spring 502 comprises a base 504 and a movable portion 506 integrated with the base. In some embodiments, the movable portion 506 moves between a collapsed state, for example as shown in FIG. 5A and an expanded state, for example as shown in FIG. 5B. In some embodiments, the expansion of the movable portion is an axial expansion along an axis that is substantially perpendicular to the base 504.

According to some exemplary embodiments, the spiral spring 502, for example the movable portion 506, has a double spiral formation. In some embodiments, the double spiral formation is formed by winding two metal parts, for example wires or strips from opposite directions around a long axis of the spiral spring until they are joined for example at an apex of the spiral spring. In some embodiments, the metal parts are joined using welding or soldering. In some embodiments, the spiral spring 502, optionally formed from a shape memory alloy, expands into a pre-deformed expanded shape, when heated to a temperature level between 37 °C and 65 °C, for example when heated to a temperature level between 37 °C and 45 °C, for example when heated to a temperature level between 40 °C and 60 °C, or any temperature level with an intermediate, smaller or larger range of temperatures.

According to some exemplary embodiments, a spiral spring, for example spiral spring 502, is formed by cutting a Niti Plate/sheet, for example by laser cutting, with a desired pattern of a flat shape coil. Then, in some embodiments, the flat shape coil is shaped to a pre-deformed shape using a mold and heat treatment at approx. 500 °C to a final shape-which then acquires a Shape memory. In some embodiments, additional options for manufacturing is by using laser cutting of a NiTi tube and/or 3D printing of NiTi.

According to some exemplary embodiments, spiral springs, for example as shown in FIG. 5D can be assembled together to form an array of spiral springs. In some embodiments, spiral springs are attached to each other, for example by at least one fastener to form the array. Alternatively, the spiral springs are attached to each other and are coupled to a shared base. Alternatively or additionally, the spiral springs are attached to each other and are coupled to a shared tissue interface configured to contact soft tissue of the body, for example skin, fat, and muscle tissue.

According to some exemplary embodiments, for example as shown in FIG. 5D, a single solid actuator, for example a spiral spring actuator, is in contact with 2 or more, for example to 2,3,4,5,6 or any larger number of solid actuators. In some embodiments, actuator 502 is in contact with actuators 508, 510, 512 and 514. In some embodiments, a geometric polygon shape of the base 504 allows contact with a plurality of actuators to the sides of the polygon, for example to form an array of actuators. In some embodiments, for example as shown in FIG. 5D, the base 504 of the actuator is shaped as a hexagon that allows a single actuator to contact to up to 6 actuators, for example via the sides of the hexagon-shaped base.

Reference is now made to FIGS. 5E and 5F depicting an implant comprising a plurality of spiral actuators coupled to an implant base and to a tissue interface, according to some exemplary embodiments of the invention.

According to some exemplary embodiments, for example as shown in FIG. 5E, an array of polygon-shaped spiral actuators, for example actuators 516, 518, 520 and 522, is coupled to an implant base 524. In some embodiments, each base of the actuators is coupled to the implant base. In some embodiments, for example as shown in FIG. 5F, an implant 530 comprises the implant base 524 a tissue interface 532 coupled to a movable portion, for example movable portion 506 of each of the actuators. In some embodiments, the tissue interface 532 is a soft tissue interface, optionally filled with a fluid, for example a viscous fluid, or a gel, for example silicon gel. In some embodiments, the tissue interface 532 is coupled to an apex of the actuators movable portion. In some embodiments, the tissue interface 532 is shaped as a cap positioned on top of the actuators.

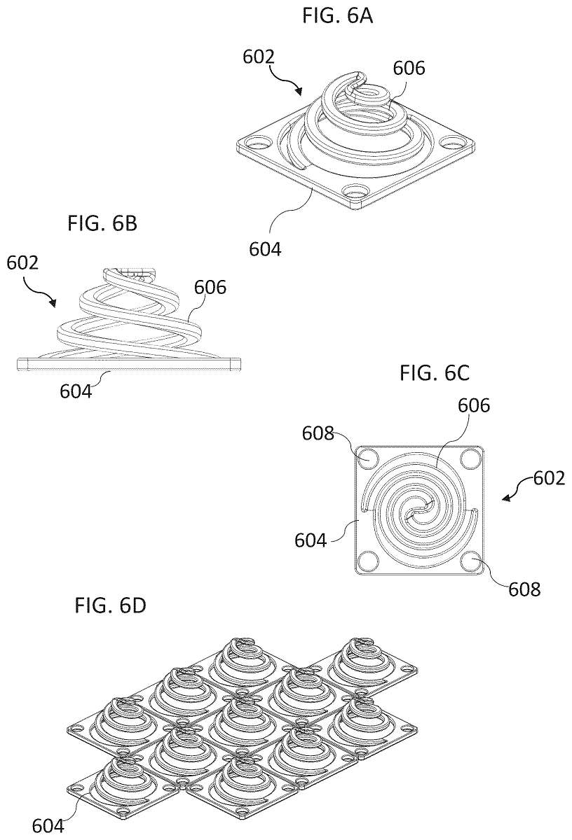

Reference is now made to FIGS. 6A-6D, depicting a solid actuator, for example an actuator spring having a rectangular base, according to some exemplary embodiments of the invention.

According to some exemplary embodiments, for example as shown in FIGS. 6A-6C, a solid actuator, for example a spring actuator 602 comprises a rectangular base 604 and a movable portion 606. In some embodiments, the movable portion 606 is integrated with the base 604. In some embodiments, the spring actuator 602 is similar to the spring actuator 502 having a hexagon shape base compared to the rectangular, for example square base of actuator 602.

According to some exemplary embodiments, for example as shown in FIG. 6D, the rectangular base 604 allows to form an array of actuators, by attaching up to 4 actuators to a single actuator, for example via sides of the actuator rectangular base. In some embodiments, for example as shown in FIG. 6C, an actuator base 604 comprises one or more openings, for example openings 608 for mechanically coupling the base 604 to a tissue interface, for example to a soft tissue interface of an implant or to a body tissue, for example to bone tissue.

### Exemplary Actuator Arrays

According to some exemplary embodiments, actuators of an implant are arranged in an array, for example to increase contact area with a soft tissue interface of an implant, or to allow, for example, efficient anchoring of the implant to bone tissue. In some embodiments, a position of each or at least some of the actuators, for example spiral actuators, in the array is fixed relative to other actuators, for example adjacent actuators, in the array. Alternatively, each or at least some of the actuators are movable, for example laterally movable, relative to other actuators in the array. In some embodiments, the actuators laterally move within the array when exposed to an energy, for example an energy that heats the actuators to a temperature higher than a specific temperature level. In some embodiments, the actuators are connected to each other by connectors that allow removal of one or more actuators form the array, for example to allow adjustment of the array or implant shape and/or size to a specific implantation site in the body.

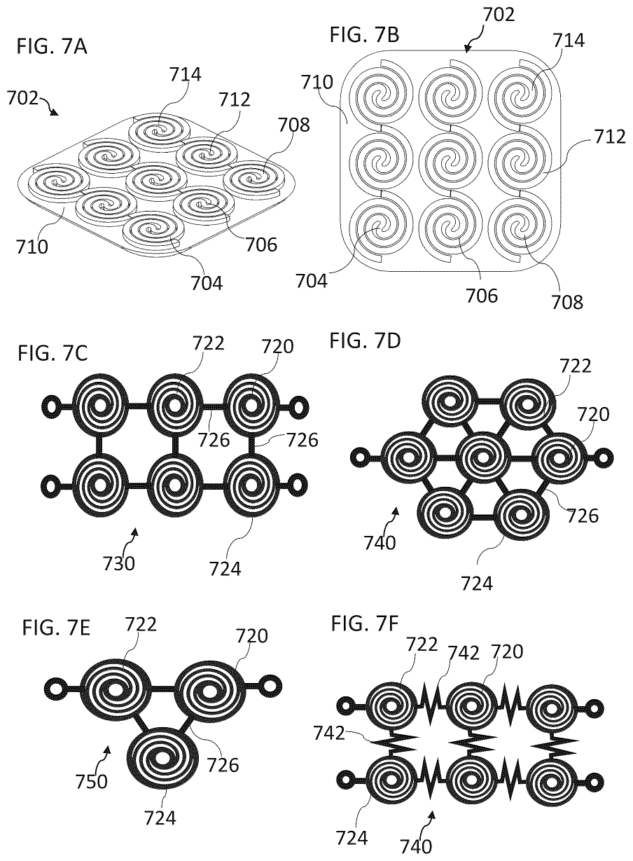

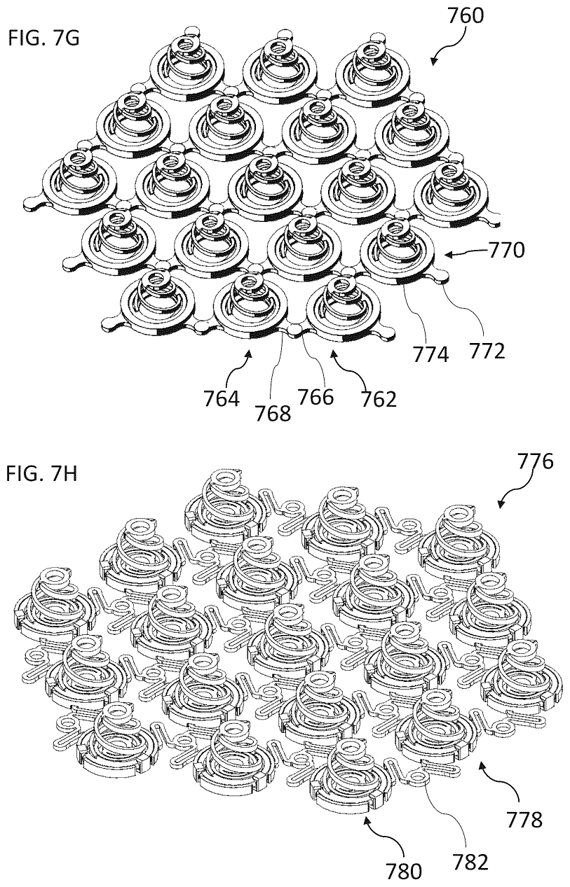

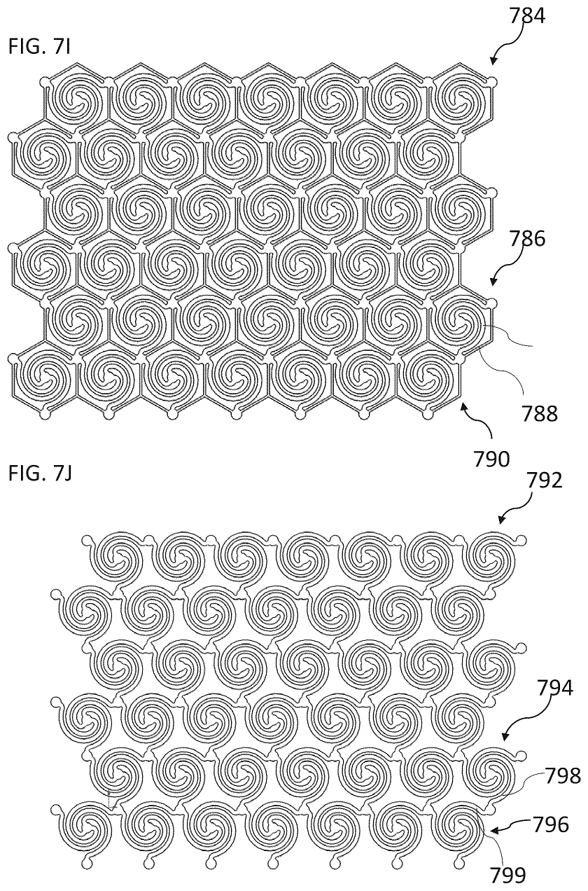

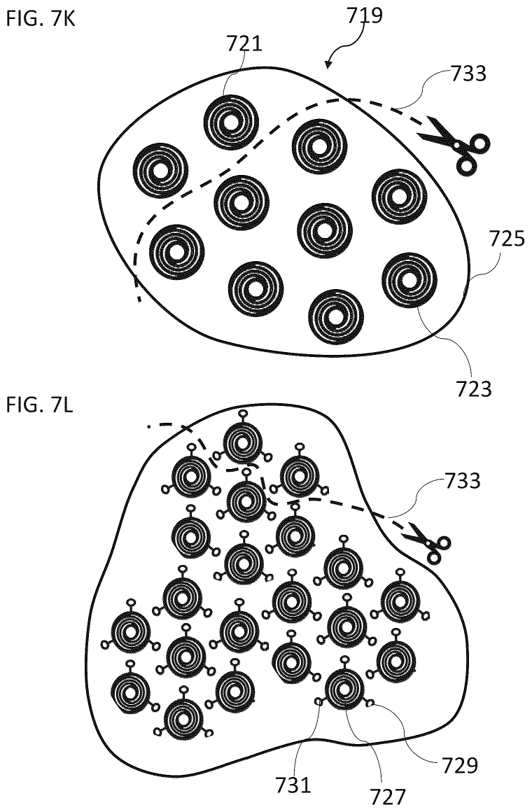

Reference is now made to FIGS. 7A and 7B, depicting an array of actuators, for example spiral actuators, coupled to each other according to some exemplary embodiments of the invention.

According to some exemplary embodiments, an implant 702 comprises a plurality of actuators, for example spiral actuators 704, 706 and 708 arranged in an array. In some embodiments, the spiral actuators 704, 706 and 708 are coupled to a base or a tissue interface 710, optionally made from a soft material, for example silicon. In some embodiments, at least some of the spiral actuators of the array, for example actuators 708, 712 and 714 are coupled to each other, for example via a spiral body or winding forming the spiral body of each spiral actuator. Optionally, two or more of the spiral actuators are formed via at least one shared winding of a material forming the spiral shape of each of the actuators. Optionally, the spiral actuators 708, 712 and 714 are arranged in a row in the array, for example a linear row, one after another.

According to some exemplary embodiments, delivering energy to the actuators, optionally causing heating of the actuators, allows relative lateral movement of actuators coupled to each other within a row of actuators. Alternatively, the delivered energy allows lateral movement of a group of actuators coupled to each other, relative to a different group of actuators in the same array or in the same implant. In some embodiments, the lateral movement of the actuators deforms, for example stretches the base or the tissue interface 710. Optionally, selectively heating and/or deforming of a single spiral actuator of a group of actuators directly coupled to each other, heats and/or deforms other spiral actuators of the array.

Reference is now made to FIGS. 7C to 7E, depicting additional actuators arrays, for example spiral actuators, coupled to each other according to some exemplary embodiments of the invention.

According to some exemplary embodiments, actuators, for example spiral actuators, are interconnected to form two dimensional (2D) arrays of actuators, for example as shown in FIGS. 7C, 7D and 7E. In some embodiments, actuators 720, 722 and 724, are interconnected by at least one connector 726, for example a wire, or a strip of material. In some embodiments, the at least one connector 726 is formed from a thermally and/or electrically isolated material. Alternatively, the at least one connector 726 is formed from a thermally and/or electrically conductive material, for example metal. In some embodiments, the actuators are interconnected via a base of each actuator using the at least one connector 726.

Reference is now made to FIG. 7F, depicting an array of actuators interconnected by deformable connectors, according to some exemplary embodiments of the invention.

According to some exemplary embodiments, at least two actuators, for example actuators 720, 722 and 724 are interconnected via at least one deformable connector, for example connector 742. In some embodiments, the at least one deformable connector 742 is configured to move between a collapsed state to an expanded state, for example when heated. In some embodiments, the at least one deformable connector 742 if formed from a shape memory material, for example a shape memory alloy or a shape memory polymer. In some embodiments, the shape memory alloy comprises Nitinol. In some embodiments, deformation of a connector 742 connecting two actuators in an array, changes a distance between the two actuators, thereby deforming the array. In some embodiments, deformation of the connectors moves the actuators in a lateral direction relative to each other.

According to some exemplary embodiments, two or more spiral solid actuators are interconnected directly, for example via bases of the actuators or via a spring or a spiral portion of the actuators. Alternatively, each actuator comprises at least two extensions, extending from the actuators, configured to allow coupling of two actuators to each other. In some embodiments, a single actuator comprises two or more extensions, for example 2, 3, 4, 5, 6 or any larger number of extensions, configured to allow coupling of the actuator to other actuators of the array.

A potential advantage of having actuators that are interconnected by extensions to form an array, may be to allow easy shaping of the array to fit a desired shape or size by disconnecting actuators from the array at the extensions, optionally by cutting, without or with minimal damage to the actuators themselves. In some embodiments, this may allow having a modular implant or a modular actuators array that can easily modified when needed.

Reference is now made to FIGS. 7G to 7J depicting arrays of interconnected spiral actuators, according to some exemplary embodiments of the invention.

According to some exemplary embodiments, for example as shown in FIG. 7G, an array 760, for example a grid, of actuators, comprises two or more actuators for example actuators 762 and 764 interconnected to each other via one or more extensions, for example extensions 766 and 768. In some embodiments, each actuator is formed with at least one extensions, configured to interconnect the actuator with at least one different actuator. In some embodiments, actuators are interconnected, for example coupled to each other, by coupling an extension of a first actuator with an extension of a second actuator. Optionally, the entire array is formed as a single unit, where all of the actuators and extensions are formed from the same material, for example a deformable material, optionally a shape memory alloy, for example as shown in FIGS. 7I and 7J. According to some exemplary embodiments, an actuator 770 comprises at least one extension, for example extension 772, extending outwardly from a base 774 of the actuator. Optionally, each actuator comprises at least 3 extensions. In some embodiments, the extensions are evenly distributed on a circumference of the actuator or the actuator base. Alternatively, the extensions are unevenly distributed on a circumference of the actuator. In some embodiments, the number of extensions of all actuators in an array is similar or varies. In some embodiments, an actuator is formed with the at least one extension. Alternatively, the at least one extension is coupled to at least one actuator after the actuator is formed, and optionally when forming the array.

According to some exemplary embodiments, the at least one extension is planar. In some embodiments, at least one extension interconnecting two actuators has at least one narrow width portion relative to a width of other portions of the extension along the extension length. Optionally, the at least one narrow width portion indicates a disconnection point of the extension, to allow separation between the two actuators, for example by cutting the extension at the narrowed portion of the extension. A potential advantage of having a narrowed portion may be to allow easy disconnection of the at least one extension, and thereby easy disconnection between two actuators. Optionally, the extensions are formed from the same material as the actuators, as a single integrated unit, for example as shown in FIGS. 71-7J.

According to some exemplary embodiments, for example as shown in FIG. 7H, an array 776 comprises two or more actuators, interconnected by at least one twisted extension 782. In some embodiments, the at least one twisted extension is formed from a deformable material, configured to deform when exposed to energy. In some embodiments, the material comprises a shape memory alloy. In some embodiments, deforming of the extension allows lateral movement of the actuators relative to each other in the array. Alternatively or additionally, deforming of the extension allows lateral elasticity of the array at the plane of the actuators.

According to some exemplary embodiments, the at least one twisted extension forms and/or indicates a disconnection region between adjacent actuators in the array.

According to some exemplary embodiments, for example as shown in FIG. 7I, an array 784 of actuators comprises actuators connected to each other via a base of each actuator, optionally formed by precision cutting, for example laser cutting. In some embodiments, the actuators are connected to each other as tiles. In some embodiments, at least some or each of the actuators, comprises a polygon-shaped base, that can be attached to other adjacent actuator bases, by at least one side of the polygon-shaped base. In some embodiments, the base is shaped as a triangle, a quadrilateral, a rectangle, a hexagon, or optionally any polygon shape that can tile. A potential advantage of having a polygon shape may be to allow a larger surface area when contacting a surface, for example a polymeric or a silicon surface onto which the actuators are coupled, to optionally prevent collapse of the surface upon expansion of the actuators at an opposite direction.

According to some exemplary embodiments, for example as shown in FIG. 7I, actuator 786 comprises a hexagon-shaped base 788 shaped and sized to be coupled to at least one actuator, for example actuator 790 via a side of the base. In some embodiments, actuator 786 is configured to be coupled to up to 6 actuators via sides of the hexagon-shaped base.

According to some exemplary embodiments, each actuator, for example actuator 786 comprises the base 788 and at least 3 spiral extensions, each having a first end coupled or integrated with the base 788 and a second end located away from the base 788. In some embodiments, the spiral extensions are twisted optionally as a helix between the first end and the second end to form a spring-like structure of the actuator. In some embodiments, the spring-like structure is configured to extend in a direction substantially perpendicular to the base, and away from the base, when the actuator or the spiral extensions are deformed. Optionally, the spiral extensions extend in response to energy applied on the actuator that optionally heat the spiral extensions.

According to some exemplary embodiments, for example as shown in FIG. 7J, an array 792 comprises two or more actuators, for example actuators 794 and 796, coupled to each other via spiral extensions of each of the actuators, for example spiral extension 798 of actuator 794 and spiral extension 799 of actuator 796. In some embodiments, the actuator comprises at least two or at least 3 spiral extensions twisted together to for a spring like extendable portion of the actuator, for example as described above with respect to actuator 786.

### Exemplary Implant Shaping

According to some exemplary embodiments, at least part of an existing implant can be adjusted to have a desired shape and/or size according to a shape and/or a size of a target implantation site in a subject body. A potential advantage of having an implant that can be shaped prior to implantation may be to allow using a small number of implants for a much larger number of implantation sites, and thereby optionally to reduce manufacturing cost while allowing maximal customization and ease of use. An additional advantage of having an implant that can be shaped prior to implantation may be that this allows to fit the implant to newly created voids in the body, for example due to trauma or tumor removal having a non-conventional shape, size and/or volume.

Reference is now made to FIGS. 7K and 7L, depicting implants that can be shaped, for example cut to shape, to have a desired shape, area, volume and/or size, according to some exemplary embodiments of the invention.

According to some exemplary embodiments, an implant comprises a plurality of actuators, for example solid actuators, coupled to a surface, for example to a base. In some embodiments, for example as shown in FIG. 7K, an implant 719 comprises a plurality, for example two or more, of actuators 721 and 723, coupled to a surface 725. In some embodiments, a base of each actuator is coupled to the shared surface 725. Alternatively, an apex of a spring portion of the actuator, for example an apex of an extendable portion of the actuator, is coupled to the surface 725. In some embodiments, the surface is a surface of a sheet material which is opposite to a tissue contacting surface of the sheet material.

According to some exemplary embodiments, the base is formed from a polymeric material, which is optionally elastic. In some embodiments, the base to which one or more of the actuators is coupled is thin, for example has a maximal thickness in a range between 0.01 mm and 10 mm, for example a thickness between 0.01 mm and 5 mm, a thickness between 0.5 mm and 3 mm, a thickness between 1 mm and 10 mm, or ay intermediate, smaller or larger value. In some embodiments, the base is bendable, for example flexible. Optionally, the base is elastic. In some embodiments, the base is formed from a material that allows cutting. Optionally, the base comprises one or more cutting lines and/or cutting regions where a thickness of the base is narrower and/or the base includes openings, to allow easy separation between portions of the base.

According to some exemplary embodiments, the actuators are distributed on the surface 725 evenly with an even distance therebetween. Alternatively or additionally, a distance between at least some of the actuators varies. In some embodiments, the sheet material extends beyond one or more of the actuators. In some embodiments, for example as shown in FIG. 7K, the actuators 721 and 723 are directly coupled to the surface 725, for example a surface of a sheet material. Alternatively, for example as shown in FIG. 7L, actuators for example actuator 727 are coupled to a surface, for example surface 725 via one or more extensions 729 and 731, extending from each or at least some of the actuators. In some embodiments, the actuators are connected to each other via one or more extensions, as shown for example in FIGS. 7G and 7H, and are coupled to the surface via the extensions. Alternatively, as shown in FIGS. 7K and 7L, the actuators are spaced-apart and separated from each other.

According to some exemplary embodiments, for example as shown in FIGS. 7K and 7L, the implant is shaped by cutting of the surface and in between actuators along line 733, for example to shape the implant according to a desired shaped, size and/or contour.

According to some exemplary embodiments, the surface of the implant to which the actuators are coupled is formed from a material that is readily cuttable, for example using scissors, a blade, a knife, or any cutting edge.

### Exemplary Implant With Spring Actuators

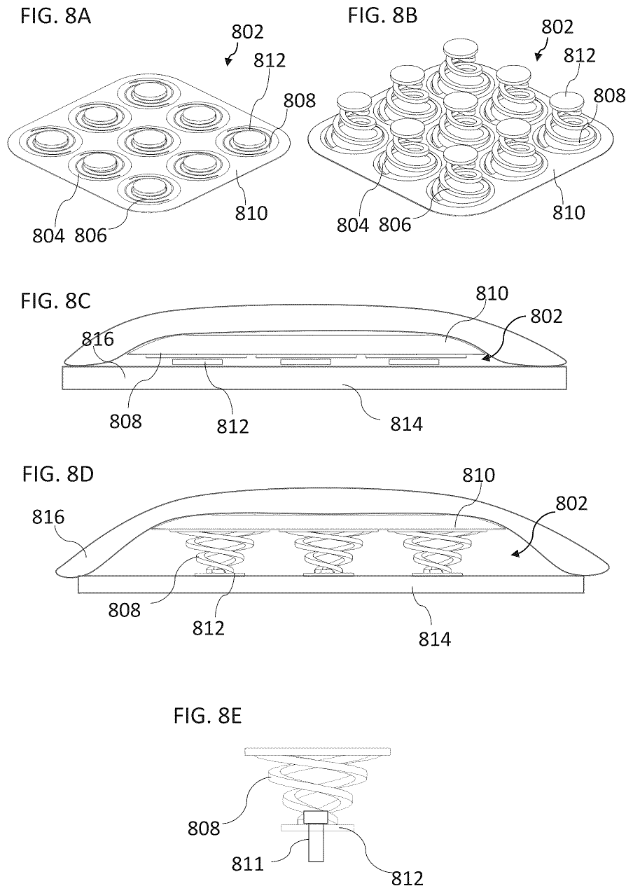

Reference is now made to FIGS. 8A-8D depicting an implant comprising an array of spring actuators, according to some exemplary embodiments of the invention.

According to some exemplary embodiments, an implant 802 comprises an array of actuators, for example spiral actuators 804, 806 and 808, coupled to a first tissue interface 810, for example a soft tissue interface. In some embodiments, each of the actuators is separately coupled to a second tissue interface, for example a rigid tissue interface. In some embodiments, the soft tissue interface is an interface between the implant, for example between one or more of the implant actuators and the soft tissue of the body, for example muscle, ligament, tendons, connective tissue, fat tissue, skin tissue. In some embodiments, the hard tissue interface is an interface between the implant, for example between one or more of the implant actuators and hard tissue of the body, for example bone tissue.

According to some exemplary embodiments, the soft tissue interface 810 comprises a soft interface, for example an inflated or an inflatable interface, a cushion. In some embodiments, the soft tissue interface is filled with fluid, for example liquid, gel, viscous gel, and/or viscous fluid. In some embodiments, the soft tissue interface 810 is configured to prevent damage to soft tissue of the bodies when one or more of the actuators of the implant push the soft tissue interface against the soft tissue of the body, for example to lift the skin surface.

According to some exemplary embodiments, the actuators 804, 806 and 808 of the implant move between a collapsed state, for example as shown in FIG. 8A, and an expanded state, for example as shown in FIG. 8B. In some embodiments, the implant 802 is introduced into the body when the actuators are in a compressed state, for example to have a thin implant capable of insertion through thin and/or small incisions into the body. In some embodiments, upon energizing of one or more of the actuators, the energized actuator expands into an expanded state. According to some exemplary embodiments, for example as shown in FIGS. 8C and 8D, the implant 802 is implanted between bone tissue 814 and soft tissue 816, for example soft tissue underneath skin tissue. In some embodiments, the tissue interface 812 us used to coupled, for example anchor the implant 802 to hard tissue, for example to bone tissue 814.

According to some exemplary embodiments, for example as shown in FIG. 8C, the implant 802 is implanted into the body in a compressed state, for example when one or more, or all of the implant actuators are compressed. In some embodiments, for example as shown in FIG. 8D, following activation, for example selective activation of at least one actuator or a group of actuators of the implant 802, the activated actuators expand and increase the distance between the bone tissue 814 and the soft tissue 816. In some embodiments, activation of the actuators lift the soft tissue 816 relative to the bone 814.

According to some exemplary embodiments, the selective activation of at least one actuator is performed by selective delivery of energy to the at least one actuator. In some embodiments, the selective delivery of energy leads to heating of the at least one actuator, and optionally moving the heated actuators to an expanded state. In some embodiments, each actuator or at least one actuator, for example prong actuator, is preformed to expand to a certain degree and/or when heated to a predetermined temperature. In some embodiments, an expansion degree of at least one actuator of an implant is different from at least one other actuator of the same implant.

According to some exemplary embodiments, for example as shown in FIG. 8E, at least one tissue interface of the actuator, for example tissue interface 812 comprises at least one opening which is shaped and sized to receive a nail or a screw 811, for example to allow fixation of the actuator and/or implant to a body tissue, for example to bone tissue.

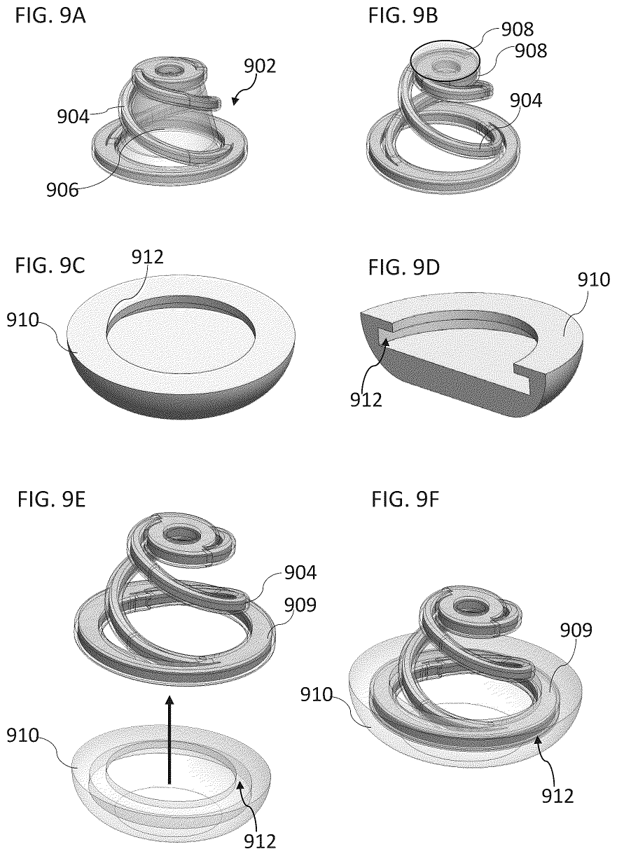

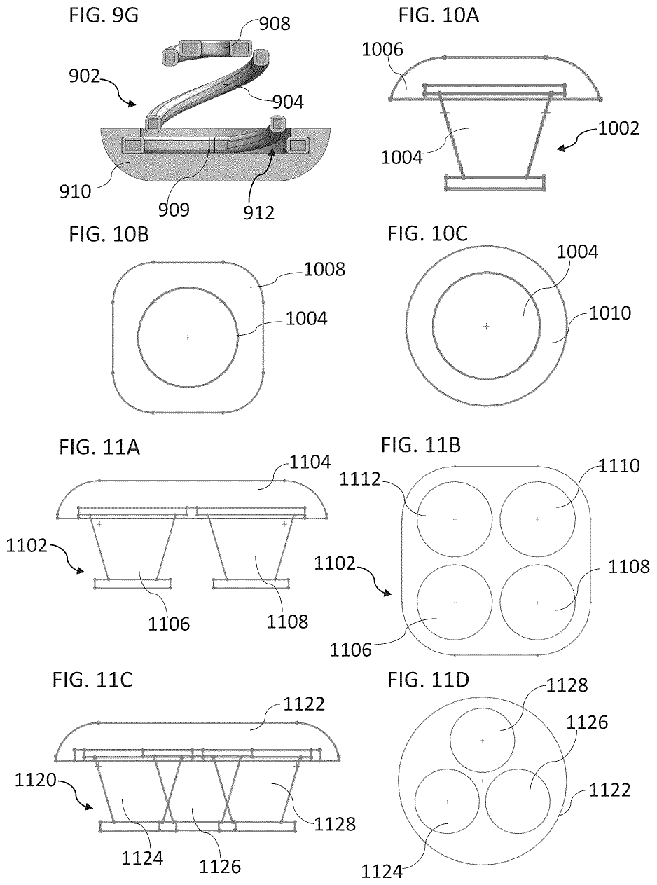

According to some exemplary embodiments, for example as shown in FIG. 9A. and 9B, an actuator is coated with a thermal isolating coating, for example a silicon coating. In some embodiments, an actuator 902, for example a spiral actuator, comprises a helical body 904 coated with a bellow 906, for example a cone-shaped bellow. In some embodiments, the bellow 906 thermally isolates the helical body, optionally formed from metal or shape memory alloy, from tissue of the body and/or from adjacent actuators of an implant. In some embodiments, for example as shown in FIG. 9B, an end 908 of the actuator body 904 is coated or coupled to a cushion, optionally filled with fluid, or a pad. In some embodiments, the end 908 is an apex, for example a narrow portion, of the actuator helical body 904.

In some embodiments, for example as shown in FIGS. 9C-9G, the implant, for example a tissue interface 910 of the implant, comprises an undercut, for example an undercut opening 912. In some embodiments, for example as shown in FIGS. 9E and 9F, the undercut is shaped and sized to receive a base 909, for example a wide end, of the body 904. In some embodiments, the undercut is shaped and sized to surround, at least partly the base 909, for example to prevent the release of the body 904 from the implant, for example from the tissue interface 910 of the implant. Optionally, tissue interface 910 is a soft tissue interface. Optionally, the tissue interface 910, is formed from a thermally insulating material, for example to thermally isolate the actuators which are optionally made from metal, from the tissue. A potential advantage of having a thermally insulating tissue interface may be to allow prevention of tissue damage, for example burns or coagulation of the tissue, when the actuators are heated.

Reference is now made to FIGS. 10A-10C depicting implants having a single actuator, according to some exemplary embodiments of the invention.

According to some exemplary embodiments, an implant 1002 comprises an actuator 1004 coupled to a tissue interface 1006, for example a soft tissue interface, optionally within an undercut of the tissue interface. In some embodiments, for example as shown in FIG. 10B, the tissue interface 1008 is a rectangular tissue interface, optionally shaped as a square. In some embodiments, the rectangular tissue interface 1008 has round corners, for example to prevent damage to the tissue contacting the tissue interface 1008. Alternatively, for example as shown in FIG. 10C, the tissue interface 1010 is round.

Reference is now made to FIGS. 11A-11D, depicting implants having an array of actuators, according to some exemplary embodiments of the invention.

According to some exemplary embodiments, for example as shown in FIGS. 11A and 11B, an implant 1102 comprises a rectangular tissue interface 1104, for example a square tissue interface, and a plurality of actuators arranged in an array of actuators, for example actuators 1106, 1108, 1110, and 1112. In some embodiments, the actuators are arranged in the array in an even number in each row or column of the array.

According to some exemplary embodiments, for example as shown in FIGS. 11C and 11D, an implant 1120 comprises a round or a triangle tissue interface 1122.

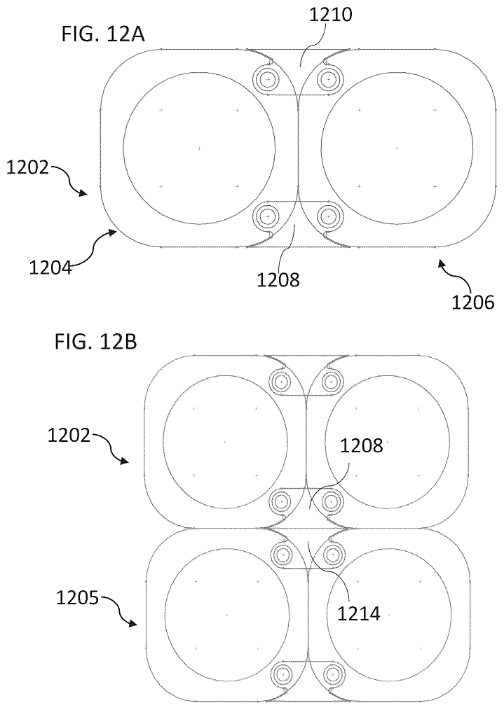

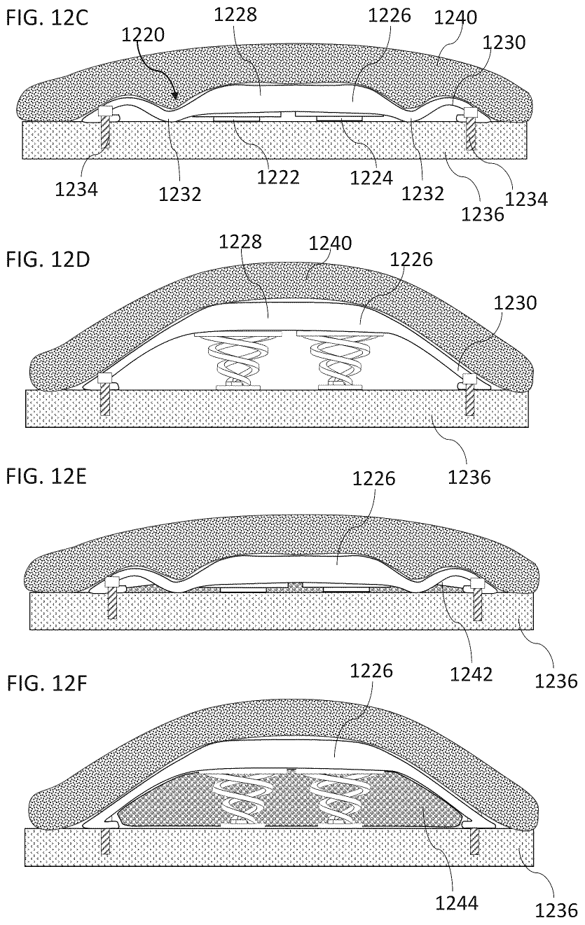

Reference is now made to FIGS. 12A and 12B, depicting poly-units implants formed from a plurality of single unit or poly units implants, according to some exemplary embodiments of the invention.

According to some exemplary embodiments, a poly unit implant 1202 comprises two single unit implants, for example implants 1204 and 1206, coupled to each other with connectors 1208 and 1210. In some embodiments, the connectors, for example connectors 1208 and 1210 at least partly fill the gaps between the two units 1204 and 1206. In some embodiments, the connectors 1208 and 1210 are formed from a soft material, for example a soft polymer, a hard material, for example a hard polymer, and/or a metal material. In some embodiments, the poly-unit implants are modular, and can comprise any number of single unit implants connected to each other by the connectors. In some embodiments, the connectors are configured to be reversibly assembled to the singe unit implants, for example to allow easy disassembly for forming poly unit implants with various shapes and sized. In some embodiments, the connectors are configured to allow generation of modular implants, for example modular multi-unit implants.

According to some exemplary embodiments, for example as shown in FIG. 12B, the connectors are configured to be coupled, for example reversibly coupled to each other. For example to allow modular formation of poly units implants. In some embodiments, implant units 1202 and 1205, are coupled to each other to form a tetra unit implant, by interconnecting connector 1208 and connector 1214. In some embodiments, connecting single units of actuators to form poly-unit implants allows, for example, to fit an implant to a shape of an implantation site. In some embodiments, the single unit have a round shape, a rectangular shape, a hexagon shape or any geometrical shape.

### Exemplary Implant With Non-collapsed Edge Portion

According to some exemplary embodiments, an implant comprises one or more actuators covered by a cover, which optionally serves as a tissue interface, configured to be placed in contact with tissue. In some embodiments, the cover comprises at least one portion at the edge of the implant that remains in a non-collapsed state, when the implant actuators are collapsed. In some embodiments, when the actuators of the implant expand, the central portion of the cover is stretched by the actuators. In some embodiments, stretching of the cover central portion aligns the cover central portion with the edge non-collapsed position to form a uniform smooth outer surface of the implant, for example at the implant edges.

Reference is now made to FIGS. 12C and 12D, depicting an implant with a non-collapsed edge portion when the implant actuators are in a collapsed state (12C) and when the implant actuators are in an expanded state (12D).

According to some exemplary embodiments, an implant 1220 comprises at least one actuator, for example actuators 1222 and 1224. In some embodiments, the implant 1220 further comprises a cover 1226, optionally formed from a soft and/or compressible and/or flexible material, for example silicon. In some embodiments, the cover comprises at least one central portion 1228, and at least one edge portion 1230. In some embodiments, the central portion 1228 is located above the actuators, and optionally contact an end of the actuators. In some embodiments, the edge portion 1230 is located at the edge of the implant 1220. In some embodiments, the central portion 1228 is thicker compared to the edge portion 1230 of the cover. Optionally, the edge portion is located in the circumference of the implant 1220. In some embodiments, the central portion is connected to the edge portion via a hinge portion 1232 of the cover 1226.

According to some exemplary embodiments, the edge portion 1230 comprises one or more openings, which are shaped and sized to receive a screw 1234 or a nail, for fixing the edge of the implant 1220 to a tissue, for example to bone tissue. Optionally, the cover edge portion 1230 is tapered.

According to some exemplary embodiments, the edge portion 1230 of the cover 1226 is non-collapsed, for example the edge portion 1230 remains in a non-collapsed state when the actuators 1222 and 1224 are in a collapsed state and the central portion 1228 is collapsed. In some embodiments, for example when the actuators expand into an expanded state, the actuators push the central portion 1226, aligning the central portion 1228 relative to the edge portion 1230, to form a uniform and smooth tissue contacting surface of the cover 1226. In some embodiments, expansion of one or more of the actuators lifts tissue 1240, for example soft tissue, contacting the cover, while keeping the implant 1220 fixed to the bone with a uniform smooth surface and an edge contacting bone tissue 1236.

According to some exemplary embodiments, the cover 1226 comprises a plurality of pores, which are shaped and sized to allow penetration of fluid and/or tissue inti the implant, for example into a void between the actuators and/or to the void between the cover 1226 and the bone 1236.

According to some exemplary embodiments, for example as shown in FIGS. 12E and 12F, the implant comprises a filler 1244 between the cover 1226 and the bone tissue 1236. In some embodiments, the filler is flexible and optionally an elastic filler, configured expand when the actuators expand, and to fill the void formed within the implant. In some embodiments, in an implant comprises a filler the cover is sealed for entrance of fluid and/or tissue into the implant. Alternatively, the cover comprises pores that allow entrance of fluid and/or tissue into the implant and into the filler 1244. In some embodiments, the filler 1244 comprises a material shaped as a sponge. Optionally, the filler 1244 is formed from a shape memory material, for example from a shape memory polymer or a shape memory material, for example from Nitinol.

### Exemplary Wave Springs Actuators

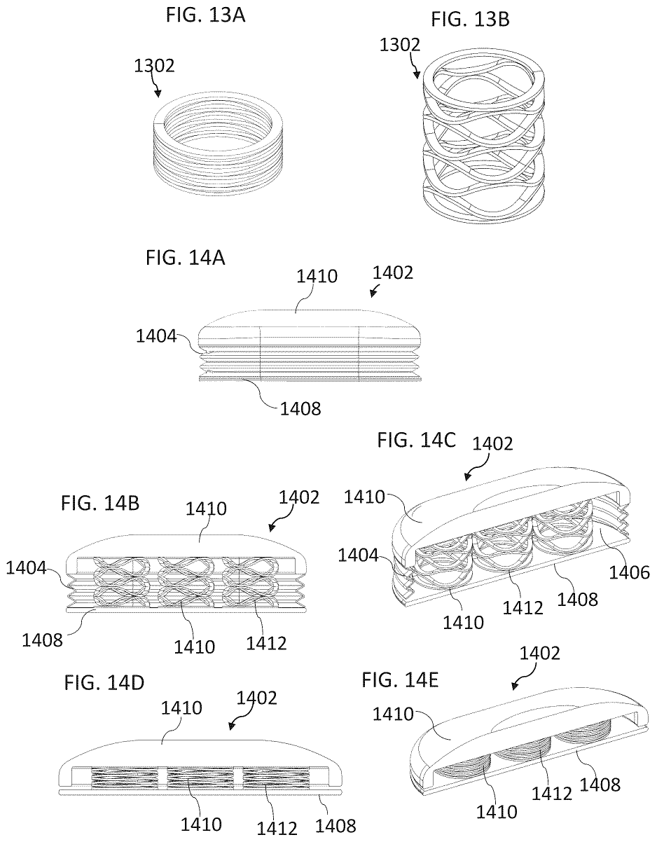

Reference is now made to FIGS. 13A-13B depicting wave springs actuators, according to some exemplary embodiments of the invention.

According to some exemplary embodiments, an implant comprises one or more wave springs actuators, for example wave spring actuator 1302. In some embodiments, the wave spring actuator 1302 is formed from a shape memory alloy, for example Nitinol (NiTi), or a copper-aluminum-nickel alloy. In some embodiments, a spring actuator formed from a shape memory alloy, for example the wave spring actuator 1302 is configured to move between a compressed state, for example a martensite state, optionally when cooled, and an expanded state, for example an austenite state, optionally when heated.

According to some exemplary embodiments, the spring actuator is configured to move into a martensite state, for example a compressed state shown in FIG. 13A, when the shape memory alloy is cooled to a temperature lower than a predetermined value, for example to a temperature lower than 38 °C, lower than 37 °C, lower than 35 °C, or any intermediate, smaller or larger temperature level. In some embodiments, the spring actuators move into a martensite state, when they are cooled and they are forced to stay in a compressed state, for example by applying an external force on the actuators. Alternatively, the spring actuators have two shape memory states, and cooling the springs is sufficient to move between a first memory state to a second memory state, without actively compressing the actuators.

According to some exemplary embodiments, the spring actuator is configured to acquire an austenite state, for example an expanded state shown in FIG. 13B, when the shape memory alloy is heated to a temperature higher than a predetermined value, for example to a temperature level higher than 40 °C, higher than 38 °C, higher than 37 °C, higher than 35 °C, or any intermediate, smaller or larger temperature level. In some embodiments, the actuator, for example the spring actuator acquires a fully expanded state when the transition from martensitic to austenitic phase is completed, meaning Af (austenite finish), to reach this Af state, the transition temperature values need to be higher (for example 50-60 °C) compared to the As (austenitic start 35-38 °C). the phase transition temperature values are predetermined according to the desired transition temperature.

In some embodiments, a plurality of spring actuators, for example wave spring actuators are arranged in an array within an implant.

Potential advantages of using wave spring actuators may be that they allow increased height, force and can be formed in different diameter sizes.

In some embodiments, a single wave spring formed from NiTi applies a compression force in an austenite state selected from a range between 700 and 1100 grams (gr), and in a martensite state, a single wave spring formed from NiTi applies a compression force selected from a range between 300 and 600 grams (gr).

In some embodiments, the actuator shape and/or size is designed according to an expansion force required to lift the tissue. In some embodiments, the force/strength of the actuator depends on the geometry of the element: width of the material and/or length of an element structure.

In some embodiments, adding more actuators to the implant proportionally increases the force distributed to the tissue. Optionally, compression force is proportional to the overall area of the implant depending on the number of actuators supporting it.

### Exemplary Enclosed Implant

According to some exemplary embodiments, an implant comprises an enclosed body having a base configured to couple the implant body to a hard tissue, for example to bone, and soft tissue interface, configured to allow contact between the implant body and soft tissue. In some embodiments, the implant comprises one or more actuators that are enclosed within the body. In some embodiments, the enclosed body is configured to isolate the actuators and/or the inner lumen of the implant from tissue of the body following implantation. In some embodiments, the implant's body is an expandable body that is configured to move between a collapsed state and an expanded state. In some embodiments, the body is configured to expand, optionally in an axial direction, for example according to the actuators alignment, following heating of the actuators.

Reference is now made to FIGS. 14A-14E depicting an enclosed expandable implant, according to some exemplary embodiments of the invention.

According to some exemplary embodiments, an implant, for example implant 1402 comprises an expandable body 1404 having an inner lumen 1406. In some embodiments, the implant 1402 comprises a base 1408 configured to attach the implant 1402 to a hard tissue, for example to bone. In some embodiments, the base is formed from a rigid material. Alternatively, the base 1408 is formed from a flexible material. In some embodiments, the body 1404 comprises a tissue interface 1410, for example a soft tissue interface. In some embodiments, the tissue interface 1410 comprises a layer of soft material, which optionally includes fluid, for example liquid, air, gas or gel. Optionally, the tissue interface layer is thick, for example thicker than the base 1408.

According to some exemplary embodiments, the implant 1402 comprises a plurality of actuators, for example actuators 1410 and 1412, optionally arranged in an array, located within the inner lumen 1406 of the implant 1402. In some embodiments, for example as shown in FIGS. 14B and 14C, the actuators are positioned and aligned between the base 1408 and an inner surface of the tissue interface 1410. In some embodiments, a first end of each actuator is coupled to the base 1408 and a second end of each actuator is coupled to the inner surface of the tissue interface 1410.

According to some exemplary embodiments, the actuators, for example actuators 1408, 1410 and 1412, are expandable actuators, comprise, for example, the wave spring actuators shown in FIGS. 13A-13B. In some embodiments, the actuators are configured to move between a collapsed state, for example as shown in FIGS. 14D and 14E and an expanded state, for example as shown in FIGS. 14B and 14C. In some embodiments, when one or more of the actuators expand, the body 1404 of the implant expands, for example as shown in FIGS. 14B and 14C. In some embodiments, when the body of the implant expands, the tissue interface 1410 pushes soft tissue and changes curvature and shape of the external surface of the skin, for example as shown in FIGS. 2D and 8C.

According to some exemplary embodiments, the body 1404 is shaped as a concertina having folds, for example to allow the body 1404 to collapse and expand.

### Exemplary Flexible Implant

According to some exemplary embodiments, an implant is flexible, for example to allow contact and/or anchoring of the implant to tissues, for example rigid tissues with a non-planar, for example curved shape. In some embodiments, rigid tissues, for example bone tissue with non-planar shape is found in the head. In some embodiments, the implant is flexible enough to be placed in contact and optionally to be anchored to skull bone.

According to some exemplary embodiments, the flexible implant is configured to be bent in one or more directions and/or along one or more axes of the implant. In some embodiments, the flexible implant comprises a flexible tissue interface and/or a flexible array of actuators.

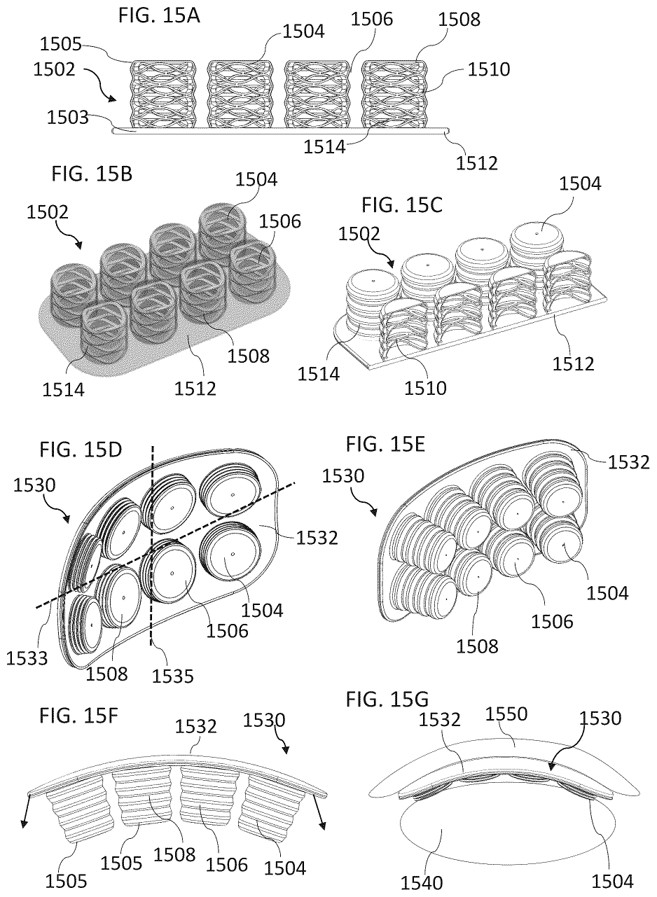

Reference is now made to FIGS. 15A-15F, depicting a flexible implant, according to some exemplary embodiments of the invention.

According to some exemplary embodiments, an implant 1502 is flexible, for example, bendable in one or more directions. In some embodiments, implant 1502 comprises a tissue interface 1512, and a plurality of actuators, for example actuators 1504, 1506 and 1508 coupled to the tissue interface. In some embodiments, for example as shown in FIG. 15A, each of the actuators comprise a first end 1503 coupled to the tissue interface 1512 and a second end 1505 located distally to the tissue interface 1512.

According to some exemplary embodiments, each of the actuators comprise a spring 1510, formed for example from a shape memory alloy, that is configured to move between a compressed state and an extended state, optionally when heated.

According to some exemplary embodiments, the actuators are distributed on a surface of the tissue interface 1512 to have even or not even distances between adjacent actuators. In some embodiments, the actuators are spaced-apart on the tissue interface surface. In some embodiments, each actuator comprises a cover 1514, for example a spring cover that isolates a spring of an actuator from adjacent actuators and/or tissue surrounding the implant. In some embodiments, the cover 1514 is flexible, and optionally comprises a bellow cover. In some embodiments, the cover is configured to stretch with the expansion of the actuator, for example when the spring in the actuator expands. In some embodiments, the bellow cover is formed from a sheet material, for example coated textile, in a folded accordion. In some embodiments, the bellow cover is shaped to fold and to unfold alongside with the movement of the actuators, for example actuator springs. In some embodiments, the bellow cover material is made from a polymer layer (silicone, polyurethane).

According to some exemplary embodiments, each of the bellow covers 1514 is optionally perforated, for example to allow ingrowth of tissue into the actuator following implantation, for example after actuator activation is finished. Alternatively, the bellow covers are impermeable to tissue. In some embodiments, the space between actuators of the implant allows ingrowth of tissue in between the actuators. In some embodiments the bellows are made of bio-degradable material, allowing tissue growth several months post implantation.

According to some exemplary embodiments, the tissue interface 1512 is soft and/or flexible. In some embodiments, the tissue interface 1512 comprises one or more layers of soft and/or flexible material. Optionally, for example as shown in FIGS. 15D-15F the tissue interface 1512 is coated or covered with the cover of the actuators, for example the tissue interface 1512 is covered or a cover of the tissue interface 1512 is integrated with the bellows cover.

According to some exemplary embodiments, for example as shown in FIGS. 15D-15G, the flexible implant, for example implant 1530 is configured to bend around a long axis 1533 and/or around a short axis 1535 of the implant. In some embodiments, for example as shown in FIG. 15E, bending of the implant 1530 changes a distance between a second end 1505 of each of the actuators, that is not coupled to the user interface, for example tissue interface 1532. In some embodiments, the flexible implant 1530 bends when one or more of the actuators is in a compressed state, for example as shown in FIG. 15D, and/or when the one or more of the actuators is in an extended state, for example as shown in FIGS. 15E and 15F.

According to some exemplary embodiments, for example as shown in FIG. 15G, the flexibility of the implant 1530 allow to the actuators to conform to a curved surface of a bone 1540, while placing the tissue interface 1532 in contact with soft tissue of the body.

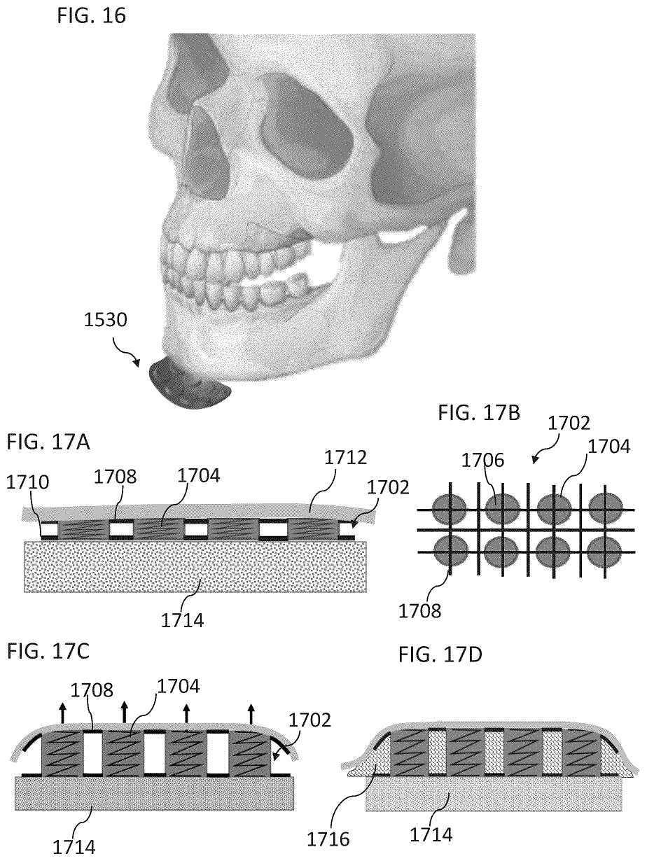

According to some exemplary embodiments, for example as shown in FIG. 16, the implant 1530 is attached to a chin bone. In some embodiments, a second end 1505 of one or more of the actuators of the implant 1530, is coupled to the chin bone, for example using adhesive, a screw or a nail.

According to some exemplary embodiments, a tissue interface such as, for example, tissue interface 312 in FIGS. 3A and 3B, includes one or more perforation that allow for tissue ingrowth through the tissue interface to one or more of the actuators. In some embodiments, the perforations are positioned to allow delivery of energy through the tissue interface to the actuators In some embodiments, the perforations are configured to provide an opening for delivery of other energy sources such as lasers that can be used to induce heat in the actuators, either selectively (for example via focused optical transmissions) or uniformly (for example via collimated or wider beam optical transmissions).

### Exemplary Mesh Implant

According to some exemplary embodiments, an implant comprises a body formed from mesh material, and one or more actuators positioned within the body. Alternatively or optionally, each of a distal end and a proximal end of an actuator is coupled to at least one layer of mesh material. In some embodiments, positioning the actuators between at least two opposite layers of mesh material allows, for example, tissue ingrowth into the implant and optionally into the actuators.

Reference is now made to FIGS. 17A-17D, depicting an implant having actuators coupled to two mesh layers, one on each side of an actuator, according to some exemplary embodiments of the invention.

According to some exemplary embodiments, an implant 1702 comprises at least one actuator, for example spring actuators 1704 and 1706, and at least two layers of mesh material, for example at least one first mesh layer 1708 and at least one second mesh layer 1710. In some embodiments, a first end of an actuator is coupled to the at least one first mesh layer 1708 of the implant, and a second end of each actuator is coupled to the at least one second mesh layer 1710 of the implant.

According to some exemplary embodiments, the implant is positioned between two tissue layers, a first tissue layer 1712, for example a soft tissue layer, and a second tissue layer 1714, for example a bone layer. In some embodiments, the mesh layer 1710 is coupled, for example anchored to the bone layer 1714, and the mesh layer 1708 is placed in contact with the soft tissue layer 1712. Optionally, for example as shown in FIG. 17A, the implant is positioned between the two layers of the body, when the actuators, or at least some of the actuators are in a collapsed state.

According to some exemplary embodiments, for example as shown in FIG. 17C, when energized, one or more of the actuators, for example actuator 1704 expands and pushes the mesh layer 1708, and the tissue, for example soft tissue 1712 contacting or leaning on the mesh layer 1708, away from the bone tissue 1714. In some embodiments, after a healing period following the implantation surgery, body tissue migrates through the mesh pores into the implant. In some embodiments, if the actuators are not coated with a coating that prevent penetration of tissue, the body tissue enters the actuators optionally before activation process is initiated. Alternatively, if the actuators are coated with a coating, the body tissue penetrates into the implant, and in between the actuators, improving and fixating the support of the actuators and improving the biological healing response with the implant, for example as shown in FIG. 17D.

In some embodiments, the mesh material comprises at least one of fabric, perforated polymer/or other biomaterial, metallic net and/or metallic mesh. In some embodiments, the mesh material allows, for example to distribute or spread a force applied by the actuator, for example spring actuator, on the tissue. Additionally or optionally, the mesh material allows, for example, tissue growth or biofluids crossing over from side to side, via the implant. Optionally, one or more or all of the actuators, are coated or encapsulated. Alternatively, one or more or all of the actuators remain uncoated.

### Exemplary Implant with Isolated Actuators

According to some exemplary embodiments, an implant comprises one or more isolating layers, configured to isolate one or more of the actuators of the implant from the surrounding environment. In some embodiments, the one or more isolating layers surround the one or more actuators. In some embodiments, the one or more isolating layers comprises folds, for example circumferential folds surrounding each of the actuators, for example to allow free expansion without interference from the isolating layer. In some embodiments, isolating the actuators of an implant allow, for example, penetration of tissue in between adjacent actuators and prevent penetration of tissue into the actuators body. Optionally, penetration of tissue into the actuator body, for example into a spring body, may interfere with movement, for example expansion and collapse, of the spring in an axial direction.

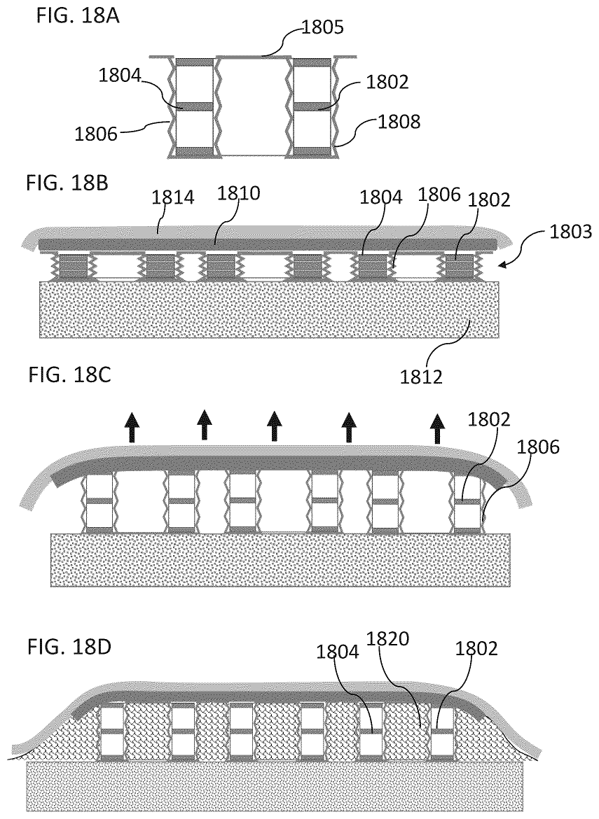

Reference is now made to FIGS. 18A-18D, depicting an implant with isolated actuators, according to some exemplary embodiments of the invention.

According to some exemplary embodiments, an implant comprises two or more spaced-apart actuators, positioned within hollow bellows, for example bellows 1806 and 1808. In some embodiments, the hollow bellows are interconnected, for example to form at least one cover or coating layer isolating the actuators from the surrounding environment. In some embodiments, the bellows comprise or are formed at least partly from silicon. In some embodiments, bellows surrounding actuators are interconnected by a sleeve, for example a silicon rubber bellow sleeve. In some embodiments, the bellows are formed from a material that is configured to prevent penetration of cells and/or tissue through the bellows, for example into the actuators. In some embodiments, the bellows are formed from a non-perforated material, or from a perforated material having pores with a size that is too narrow to allow penetration of cells. In some embodiments, the pores have pores with a maximal width value in a range between 30 μm (microns) and 5 microns, for example 30 microns and 10 microns, 15 microns and 3 microns, or any intermediate, smaller or larger range of values. In some embodiments, the pores have a maximal width value in a range between 0.1 mm and 1 mm, for example 0.1 mm and 0.4 mm, 0.6 mm and 0.9 mm, or any intermediate, smaller or larger range of values In some embodiments, the bellows are formed from material that prevents tissue outgrowth or ingrowth on the bellows.

According to some exemplary embodiments, for example as shown in FIGS. 18B-18D, an implant 1803 comprises a tissue contacting layer 1810, for example a soft tissue contacting layer. In some embodiments, the tissue contacting layer 1810 is a tissue interface of the implant, optionally formed from silicon and/or rubber. In some embodiments, for example as shown in FIG. 18B, the implant is implanted between two tissue layers, for example between bone tissue 1812 and a softer tissue 1814. In some embodiments, in a collapsed state the bellows, having circumferential folds are folded. In some embodiments, following actuators energizing, the actuator 1802 expands within the bellow 1806. In some embodiments, expansion of the actuator 1802 unfolds the below 1806, for example straightens the circumferential bellow folds.

According to some exemplary embodiments, for example as shown in FIG. 18D the bellows define a void between the isolated actuators, that allows tissue to penetrate in between adjacent isolated actuators into the implant, for example into the implant body.

According to some exemplary embodiments, the bellows are configured to thermally isolate the actuators from the surrounding environment, for example to prevent heat loss to the surrounding environment following actuators energizing. Additionally or alternatively, the bellows are configured to reduce air volume, and/or to reduce vacuum pressure on the spring actuators.

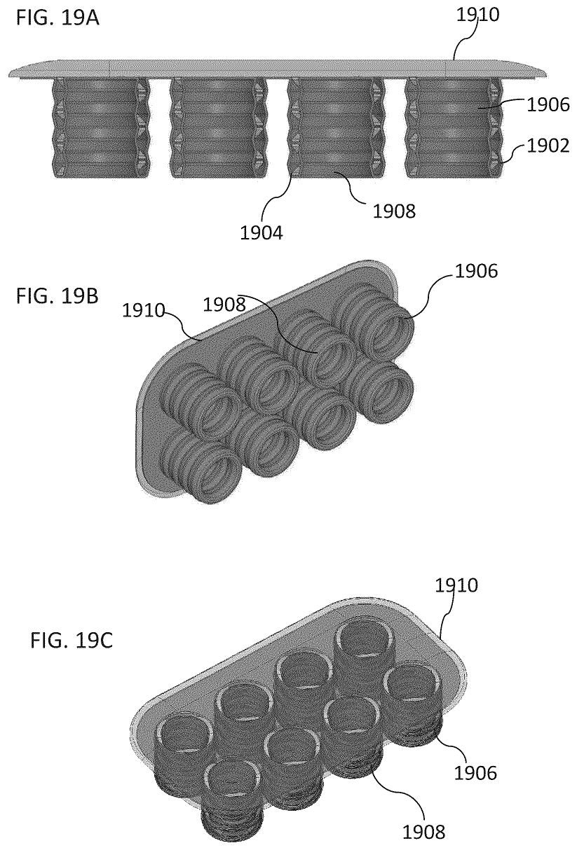

According to some exemplary embodiments, for example as shown in FIGS. 19A-19C, spring actuators of an implant, for example spring actuators 1902 and 1904 are positioned within hollowed bellows 1906 and 1908, respectively. In some embodiments, the hollowed bellows are configured to reduce folding volume, and are optionally formed from silicone or a different polymer.

According to some exemplary embodiments, the hollowed bellows 1906 and 1908, and/or the spring actuators 1902 and 1904 are coupled to an interface, for example cover 1910. In some embodiments, the cover is optionally formed from a thermal-isolating material, configured to reduce heat transfer to surrounding tissue and/or to reduce heat loss from the heated spring actuators following energizing of the spring actuators. Optionally, the hollow bellows 1906 and 1908 are integrated with the cover 1910. Optionally, the cover 1910 has a foldable shape.

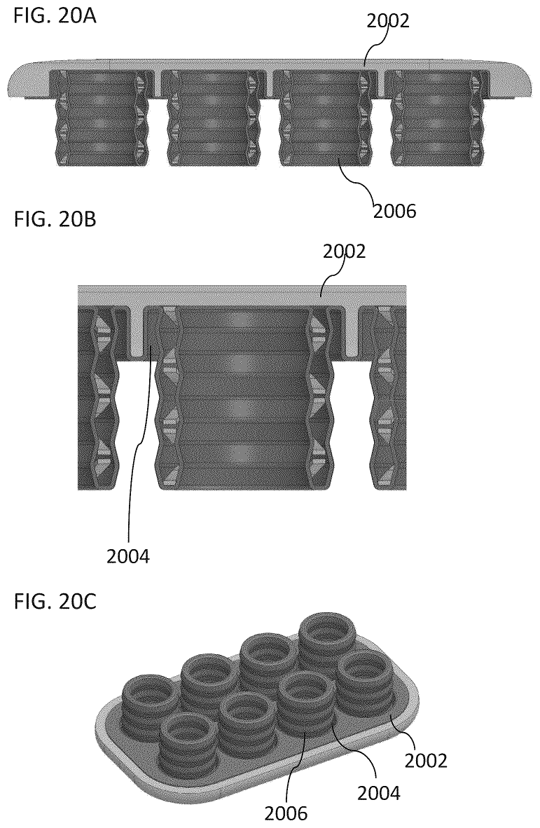

According to some exemplary embodiments, for example as shown in FIGS. 20A-20C, the interface, for example cover 2002 comprises at least one socket 2004. In some embodiments, the at least one socket 2004 is located in a surface of the cover that is opposite to a tissue contacting surface of the cover 2002. In some embodiments, the at least one socket is configured to receive at least one of, a hollow bellow and a spring actuator within the bellow.

In some embodiments, when the spring actuator is in a compressed state, for example when inserting the implant into the body, the spring does not extend out from the at least one socket, for example to maintain a thin cross section of the implant that does not interfere with the insertion of the implant into the body through a thin incision and/or with the implantation of the implant at an implantation site.

### Exemplary Expansion Actuator

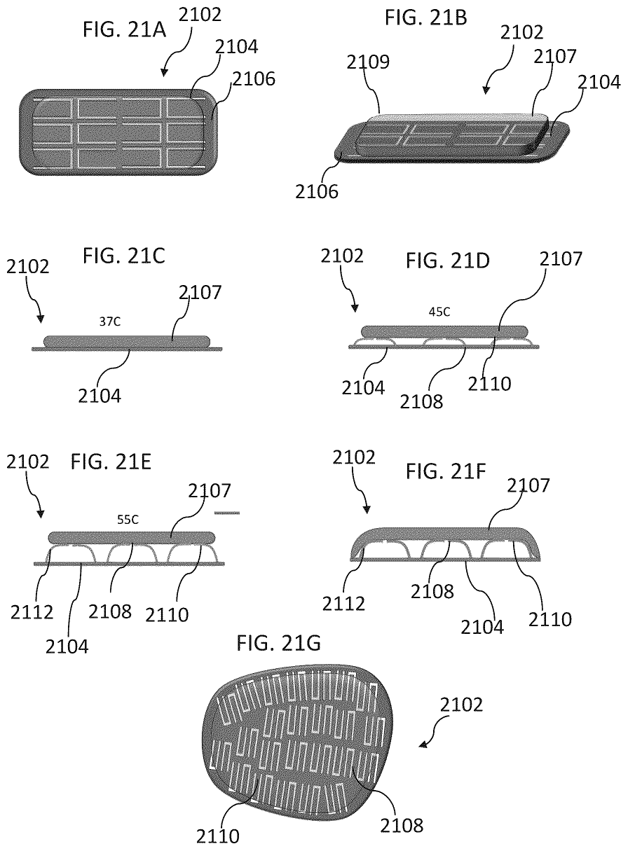

Reference is now made to FIGS. 21A-21G, depicting an implant with at least one thin expandable actuator, according to some exemplary embodiments.

According to some exemplary embodiments, an implant, for example implant 2102 comprises at least one thin expandable actuator 2104, shaped as a thin plate or a thin grid. In some embodiments, the thin expandable actuator 2104 is formed from a shape memory alloy, for example Nitinol. In some embodiments, the thin expandable actuator is located between a base 2106 and a cover 2107 of the implant 2102, optionally surrounding the thin expandable actuator. In some embodiments, the cover is a tissue contacting interface, and is optionally soft.

According to some exemplary embodiments, the thin expandable actuator comprises a plurality of expandable segments, for example bridges 2108, 2110 and 2112. In some embodiments, the bridges are configured to move between a collapsed state to an expanded state, when energized, for example when heated. In some embodiments, in a collapsed state, for example as shown in FIG. 21C, the bridges are compressed, and the actuator is flat, optionally planar. In some embodiments, when the bridges are in a compressed state, the implant 2102 has a thin cross-section, which optionally allows insertion of the implant into the body through thin or narrow incisions.

In some embodiments, a thickness of the implant 2102 in a collapsed state is within a range of 0.5mm-3 mm, for example 0.5 mm- 1 mm, 0.5 mm-2 mm, 1 mm-2 mm, 1 mm-3 mm or any intermediate, smaller or larger value or range of values.

According to some exemplary embodiments, for example as shown in FIGS. 21C to 21E, when heated, the bridges are expanded. In some embodiments, the expansion amount of the bridges depends on the temperature of the thin expandable actuator 2104, and/or the temperature of each of the bridges. In some embodiments, the bridges expand uniformly, for example when the bridges are thermally connected and/or have similar properties. Alternatively, at least one of the bridges is preformed to expand to a different degree from one or more other bridges in the implant, which may lead to a non-uniform expansion of the implant 2102. Alternatively, at least one of the bridges is thermally isolated from other bridges, and may be heated to a different temperature of other bridges, leading to non-uniform expansion of the implant 2102.

According to some exemplary embodiments, for example as shown in FIG. 21F, the bridges are encapsulated within the implant 2102, for example by the cover 2107 According to some exemplary embodiments, for example as shown in FIG. 21G, the bridges 2108 and 2110 are formed by laser cutting, and optionally can be formed in any shape and/or size.

### Exemplary Inflated Implants

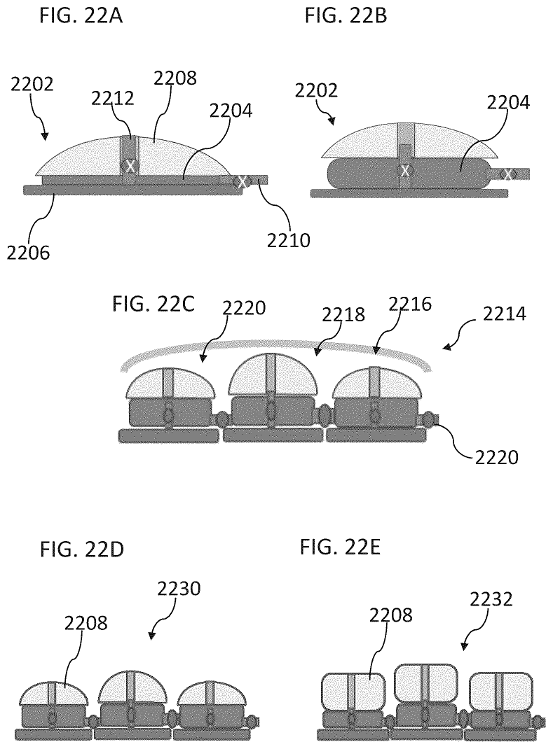

Reference is now made to FIGS. 22A-22E depicting inflatable actuators, according to some exemplary embodiments of the invention.

According to some exemplary embodiments, an implant comprises one or more inflatable actuators, for example an inflatable actuator 2202. In some embodiments, the inflatable actuator 2202, comprises at least one inflatable chamber 2204, positioned between a base 2206, for example a first tissue contacting interface, and a second tissue contacting interface 2208. In some embodiments, the base 2206 is optionally planar, and is configured to be placed in contact with tissue of the body, for example with bone. In some embodiments, the interface 2208 comprises a compressible and optionally a soft interface, for example a cushion, configured to be pushed against soft tissue of the body when the implant expands.

According to some exemplary embodiments, the implant 2202 comprises at least one channel, for example a channel 2210 coupled to the chamber 2204, and configured to allow fluid flow between the chamber 2204 and at least one fluid source. Optionally, the channel, for example channel 2212 crosses through the interface 2208. Optionally, the at least one channel, for example channels 2210 and 2212 comprises at least one valve, for example a one way valve, to control the fluid flow through the channel.

According to some exemplary embodiments, for example as shown in FIG. 22B, inflation of the chamber 2204, for example by introducing fluid into the chamber 2204 via the channel, expands the implant 2202.

According to some exemplary embodiments, for example as shown in FIG. 22C an implant, for example implant 2214 comprises a series of inflatable actuators, for example actuators 2216, 2218 and 2220. In some embodiments, the series of actuators are fluidically connected by at least one channel. In some embodiments, the at least one channel comprises at least one valve, for controlling the flow of liquid towards and/or from a single chamber of at least one inflatable actuator of an implant, or from all chambers of inflatable actuators of the implant. Optionally, each inflatable actuator comprises a fluidically separate channel to at least one inflatable chamber of the actuator, for example to allow separate inflation of the at least one inflatable chamber.

According to some exemplary embodiments, for example as shown in FIG. 22D, one or more of the tissue contacting interface 2208, of an implant has a round and/or curved external surface facing tissue, for example a round and/or a curved tissue contacting surface. In some embodiments, for example as shown in FIG. 22D, the interface 2208 is shaped as a dome. Alternatively, for example as shown in FIG. 22E, the interface 2208 of implant 2232 has a rectangular shape. Optionally, the interface 2208, for example as shown in FIG. 22E, has a flat tissue contacting or outer surface. Optionally, the interface 2208, for example as shown in FIG. 22E, has a planar tissue contacting or outer surface.

### Exemplary Modular Implant

According to some exemplary embodiments, an implant comprises two or more, for example a plurality of single actuators coupled to each other. In some embodiments, coupling of single actuators allows, for example to generate modular implant having a predetermined size and/or shape. Additionally, or alternatively, coupling of single actuators allows to form an implant that matches a selected implantation site in the body.

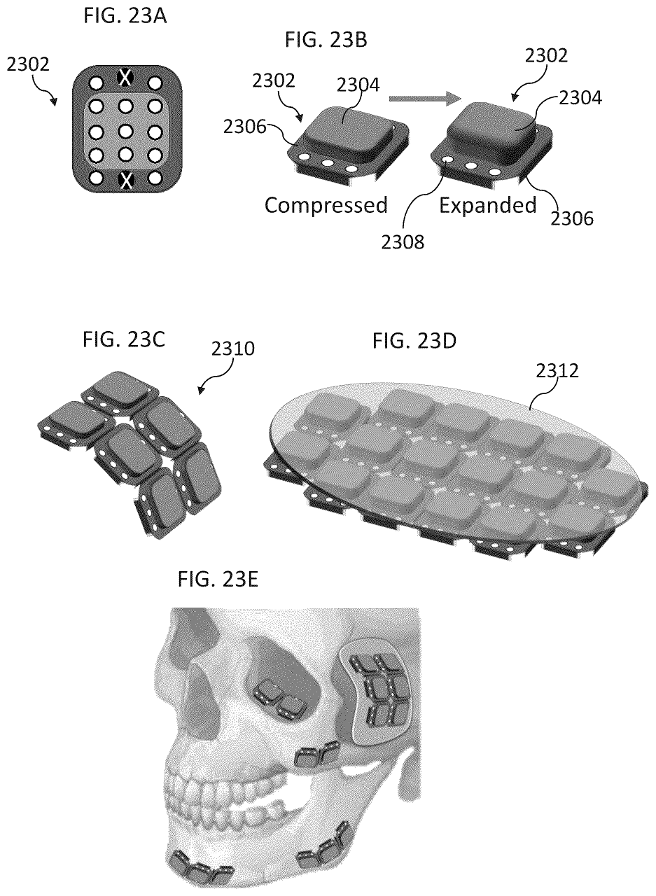

Reference is now made to FIGS. 23A-23E depicting a modular implant, according to some exemplary embodiments of the invention.

According to some exemplary embodiments, an implant, for example a modular implant comprises two or more single unit expandable actuators, for example actuator 2302. In some embodiments, for example as shown in FIG. 23B, the actuator 2302 comprises at least one expandable portion, for example an expandable chamber 2304 that is configured to move between a compressed state and an expanded state, when energized or inflated. In some embodiments, the chamber is coupled to a base 2306. Optionally, the base 2306 is shaped as a plate. Optionally the base has a customized 3D shape to fit/replace/accommodate a specific anatomical bone shape. Optionally, the base 2306 is rigid.

According to some exemplary embodiments, the base 2306 comprises one or more openings or apertures, for example opening 2308, configured to allow coupling of the base 230 to bone tissue. Additionally, the base comprises one or more openings or apertures configured to allow coupling of a base of at least one first actuator unit to a base of at least one second actuator unit. In some embodiment, at least one first actuator unit is coupled to at least one second actuator unit via a hinge. In some embodiments, the at least one first actuator unit is movably coupled to the at least one second actuator unit, for example to allow movement of the first actuator unit relative to the second actuator unit.

According to some exemplary embodiments, for example as shown in FIG. 23C, an implant, for example a modular implant comprises an array 2310 of actuator units that can be assembled or disassembled from the array. In some embodiments, for example as shown in FIG. 23D, the array is positioned between a first tissue, for example a bone tissue, and a second tissue, for example soft tissue, such that the chamber 2304 is in contact with the soft tissue.

Alternatively, an implant comprises the array of single actuator units, coupled to a single tissue interface, or two or more tissue interfaces each covers two or more actuators.

According to some exemplary embodiments, for example as shown ion FIG. 23E, the array of actuator units that are movable with respect to adjacent actuators in the array, is flexible, for example bendable, for example to fit a curvature of a tissue in the body in an implantation site. In some embodiments, for example as shown in FIG. 23E, the flexible array bends to fit a curvature of skull tissue, for example in a chin region of the head, in a temple region of the head or in the cheek region of the head.

### Exemplary Actuators Array

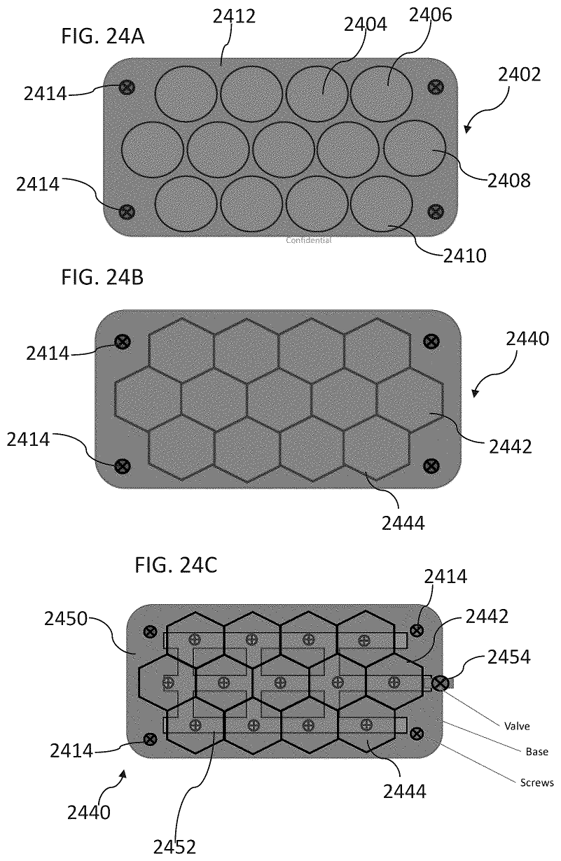

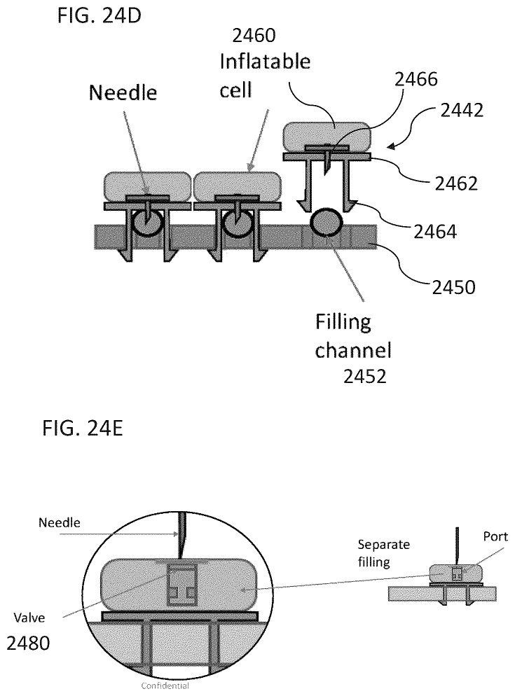

According to some exemplary embodiments, for example as described earlier in FIGS. 23C and 23D, an implant comprises an array of actuators. In some embodiments, the actuators in an array are assembled to each other. Alternatively, actuators of an array are coupled to a base layer. Reference is now made to FIGS. 24A and 24B, depicting an array of actuators coupled to a base, according to some exemplary embodiments of the invention.

According to some exemplary embodiments, for example as shown in FIG. 24A, an implant 24A comprises an array of spaced-apart actuators, for example actuators 2404, 2406, 2408 and 2410 coupled to a single base structure 2412. In some embodiments, the base, for example base 2412 comprises one or more openings or fasteners, for example screws 2414, configured to fasten the base 2412 to a body tissue, for example to bone tissue. In some embodiments, for example as shown in FIG. 24B, implant 2440 comprises a plurality of actuators, for example actuators 2442 and 2444, arranged in an array, for example a compact array. In some embodiments, the actuators 2442 and 2444 in the array contact each other, and optionally have a polygon shape that allows to have a compact arrangement of actuators that contact each other via the sides of the polygon shape.

According to some exemplary embodiments, for example as shown in FIG. 24C, a base 2450 of the implant 2440 comprises one or more fluid flow paths, for example channel 2452. In some embodiments, the implant 2440 comprises at least one valve 2454 on said channel 2452, configure to control flow into and/or out from the channel 2452. Optionally, the valve 2454 is a one-way valve. Optionally, the valve 2454 comprises a check valve.

According to some exemplary embodiments, each of the actuators, for example actuators 2442 and 2444 is configured to be assembled to the base 2450. Optionally, the actuators are configured to be irreversibly assembled to the base 2450. Alternatively, the actuators are configure to be reversibly assembled to the base 2450.

According to some exemplary embodiments, for example as shown in FIG. 24D, the actuator 2442 comprises at least one inflatable cell 2460 coupled to an actuator base 2462. In some embodiments, the actuator base is rigid and optionally comprises one or more fasteners 2464 configured to fasten the actuator 2442 to the implant base 2450. In some embodiments, each actuator comprises a puncturing element, for example a needle 2466, fluidically coupled to the cell 2460. In some embodiments, for example as shown in FIG. 24, fastening of the actuator 2442, for example using fastener 2464, inserts the needle end into the channel 2452, forming a flow a flow path between the channel 2452 and the inflatable cell 2460. In some embodiments, for example as shown in FIG. 24D, the inflatable cells of the implant are inflated via the channel 2452 in the implant base to which each of the actuators is fluidically coupled.

Alternatively or additionally, for example as shown in FIG. 24E, each of the cells of the actuators comprises at least one valve 2480, for example to allow separate inflation of each cell from other cells of the implant actuators.

### Exemplary Actuators Assembly

According to some exemplary embodiments, an implant is modular, and can be assembled upon request, for example as described at block 382 of FIG. 3D. In some embodiments, the implant is assembled by placing a desired number of actuators, for example the spiral, spring-like actuators shown in FIGS. 5A-5D, 6A-6D and 7A-7J, at selected locations, using an actuators-positioning base. In some embodiments, the base is a layer of material, for example a sheet.

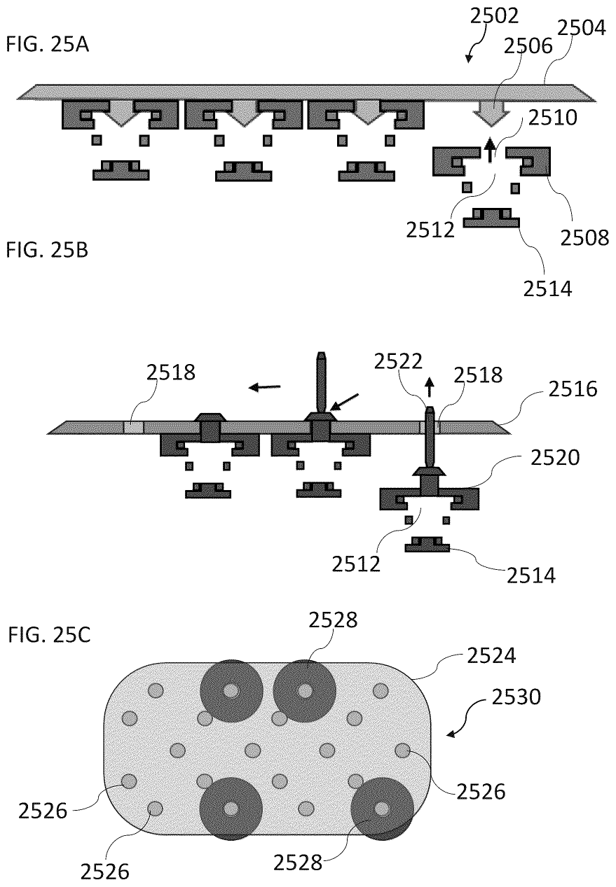

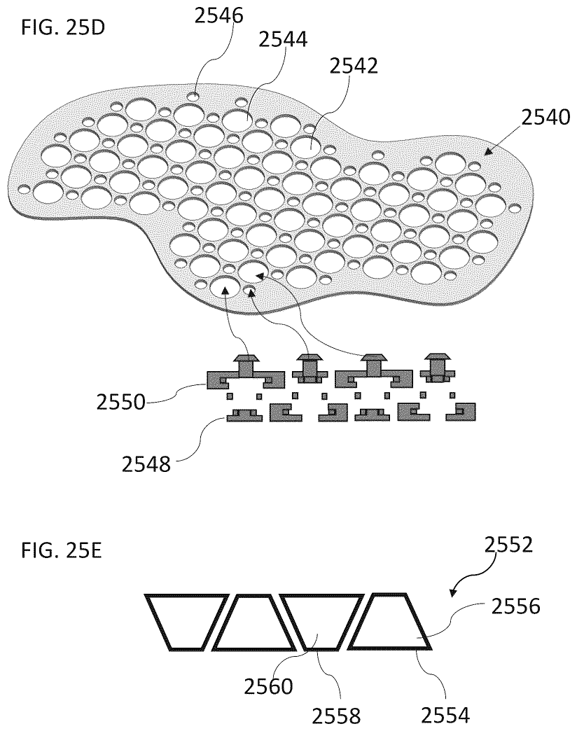

Reference is now made to FIGS. 25A-25C, depicting positioning of actuators using an actuators-positioning base, according to some exemplary embodiments of the invention.

According to some exemplary embodiments, an actuators assembly 2502 comprises a positioning base, for example layer 2504 having at least one, for example at least two predetermined positioning locations distributed in the layer 2504. In some embodiments, each positioning location is configured to couple directly or indirectly, for example via an adaptor, at least one actuator to the layer 2504. In some embodiments, for example as shown in FIG. 25A, each positioning location comprises an extension 2506, for example a pin, extending from the layer 2504. In some embodiments, an actuator adaptor, for example adaptor 2508, is configured to be coupled to the extension 2508. Additionally, the adaptor is configured to be coupled to an actuator, for example a spiral spring-like actuator. In some embodiments, each adaptor comprises a first opening 2510 which is shaped and sized to receive the extension 2506, and a second opening 2512 shaped and sized to receive an actuator 2514. Alternatively, the actuator is directly coupled to the extension, for example via an opening in the actuator.

According to some exemplary embodiments, for example as shown in FIG. 25B, the base 2516 comprises an opening 2518, at each positioning location. In some embodiments, the openings are configured to receive a portion of an adaptor, for example a pin 2522. In some embodiments, each adaptor, for example as already shown in FIG. 25A, is configured to be coupled to an actuator 2514, optionally via an opening at the adaptor.

According to some exemplary embodiments, for example as shown in FIGS. 25A and 25B, each actuator is positioned at a desired location on the base using an adaptor that is coupled to the base using, for example a snap fit mechanism, and at a specific predetermined locations in the base. Optionally, each actuator is coupled to the adaptor reversibly or irreversible, for example using a snap-fit or an interference lock. Optionally, each adaptor is coupled to the base reversibly or irreversible, for example using the snap-fit mechanism or an interference lock. Optionally, an actuator is directly coupled reversibly or irreversibly to the base, for example using a snap-fit or an interference locking mechanism.

A potential advantage of using an adaptor for coupling an actuator to a base, may be that it allows coupling of standard actuators to a base or actuators with different size of bases and extendable portions heights that can be expanded, for example at different temperatures.

According to some exemplary embodiments, for example as shown in FIG. 25C, having a base 2524 with predetermined actuator coupling locations, allows to position actuators at specific locations, and at a specific distribution on the base 2524, via adaptors 2528 or directly, to form a customized assembly 2530.

According to some exemplary embodiments, a base, is flexible, for example bendable, and includes a plurality of coupling regions, each is configured to couple at least one actuator to the base. In some embodiments, the base is formed from at least one of, polymeric material, silicon or derivatives, plastic and/or metal. A potential advantage of having a base that is formed from a different material than the actuators, for example from a polymeric material, may be to allow easy cutting of the base without harming the actuators. In some embodiments, for example as shown in FIG. 25D, base 2540 comprises a plurality of spaced-apart openings, for example openings 2542 and 2544. In some embodiments, the openings 2542 and 2544 are shaped and sized to allow coupling of an actuator 2548 to the base 2540, optionally via an adaptor 2550. In some embodiments, the actuator 2548 or the actuator adaptor 2550 is configured to be couple via snap connection to the base 2540, optionally using at least one snap-fit connector. Optionally, an actuator comprises a portion that is configured to be coupled to the base via snap connection.

According to some exemplary embodiments, the base 2540 comprises at least two type of openings, one type is for coupling an adaptor or an actuator at a first orientation to the base, for example for coupling of an actuator base directly or indirectly to the base 2540, and another type of openings, for example opening 2546, for coupling of an adaptor or an actuator at a second, optionally opposite, orientation.

According to some exemplary embodiments, for example as shown in FIG. 25E, coupling of actuators at opposite orientations to the base, allows a head to tail arrangement, in which a base 2554 of at least one first actuator 2556 is adjacent to an apex 2558 of an extendable portion of at least one second actuator 2580. A potential advantage of arranging actuators in a head to tail orientation may be to have an efficient usage of a space of a base to allow coupling of as many as possible actuators to a single shared base.

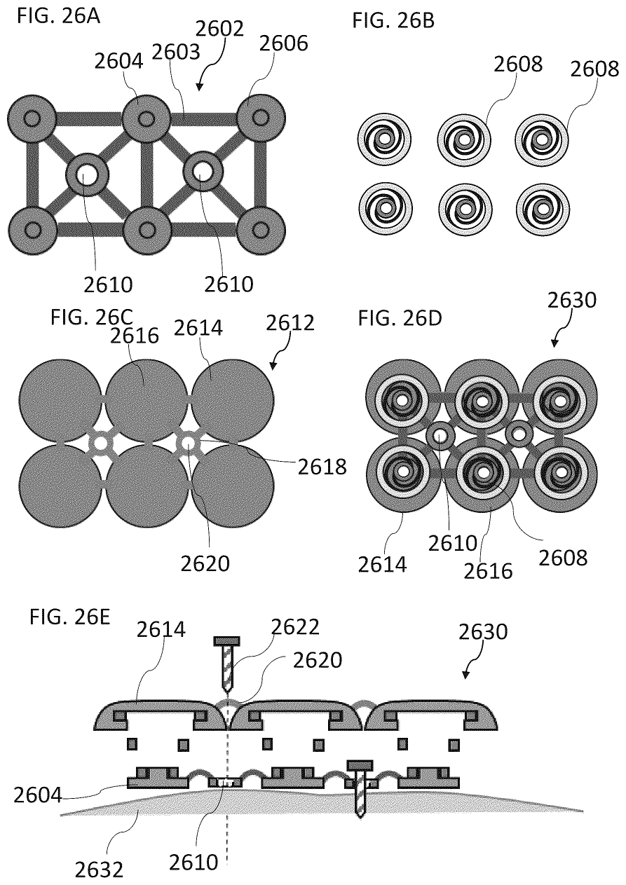

Reference is now made to FIGS. 26A-26E, depicting an implant assembly, according to some exemplary embodiments of the invention.

According to some exemplary embodiments, an implant assembly comprises an actuators positioning base, for example base 2602. In some embodiments, base 2602 is a grid comprising one or more actuator couplers, for example connectors 2604 and 2606, each is configured to be coupled to an actuator, for example a solid, spiral shaped actuator 2608. In some embodiments, each actuator is reversibly or irreversibly coupled to the base 2602, for example via the connectors 2604 and/or 2606. In some embodiments, the base 2602 comprises one or more tissue fixators, for coupling of the base 2602 to a body tissue, for example a rigid body tissue optionally comprises bone tissue. In some embodiments, the one or more tissue fixators comprises openings 2610, which are shaped and sized to receive a screw for coupling of the base to a tissue.

According to some exemplary embodiments, base 2602 is shaped as a grid, where the actuator couplers 2604 and 2606 are connected to each other via at least one bridge 2603. In some embodiments, a shape of the base 2602 can be modified by cutting or disconnecting the bridges.

According to some exemplary embodiments, the assembly comprises an array of caps, for example array 2612 comprises at least one, or a plurality of caps, for example caps 2614 and 2616. In some embodiments, the caps are spaced-apart from each other. In some embodiments, a distance between centers of adjacent caps is similar to a distance between centers of adjacent actuator couplers.

According to some exemplary embodiments, a concave side of each cap, for example a dome is configured to be coupled to an actuator, and an opposite convex side of the cap is optionally configured to be placed in contact with soft tissue. In some embodiments, adjacent caps in the caps array are interconnected to each other via one or more interconnecting bridges or interconnection portions 2618. In some embodiments, the caps array comprises one or more connectors for connecting the caps array to the actuators array. Optionally, the one or more connectors comprise openings 2620, which are aligned with openings 2610 when the caps array 2612 is coupled to the actuators base 2602. In some embodiments, for example as shown in FIG. 26E, openings 2610 and 2620 allow to insert a screw 2622 across the implant 2630 to a rigid tissue of the body, for example bone 2632.

FIG. 26D depicts the implant assembly 2630 from a bottom view.

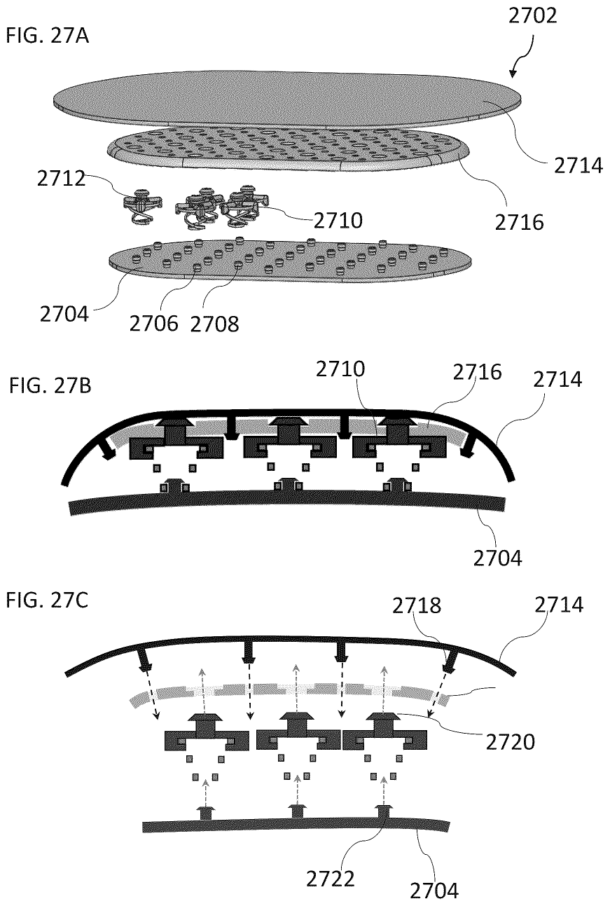

Reference is now made to FIGS. 27A-27C, depicting an implant assembly where actuators are sandwiched between a base layer and a cover layer, where the actuators interlock with the base layer and the cover layer, according to some exemplary embodiments of the invention.

According to some exemplary embodiments, an implant assembly 2702 comprises an actuators base 2704 comprising one or more actuators coupling locations 2706 and 2708, and one or more actuators or actuators housing 2710 and 2712. In some embodiments, an actuator housing comprises an actuator coupled to an adaptor, optionally irreversible.

According to some exemplary embodiments, the implant assembly 2702 comprises a cover 2714. In some embodiments, the actuators, for example actuators housing or adaptors, are coupled between the base 2704 and the cover 2714. In some embodiments, for example as shown in FIGS. 27A-27C, the cover layer 2714 is coupled to the actuators via an intermediate coupling layer 2716. In some embodiments, the actuators 2710 and 2712 and the cover 2714 interlock with the intermediate coupling layer 2716 via opposite sides, optionally using an interlocking mechanism, for example one or more snap-fit connectors. Optionally, the actuators or actuators housing interlock with the base 2704 via an interlocking mechanism, for example an interlocking connector 2722, for example a snap-fit connector.

### Exemplary Implant with an Inner Actuators Array

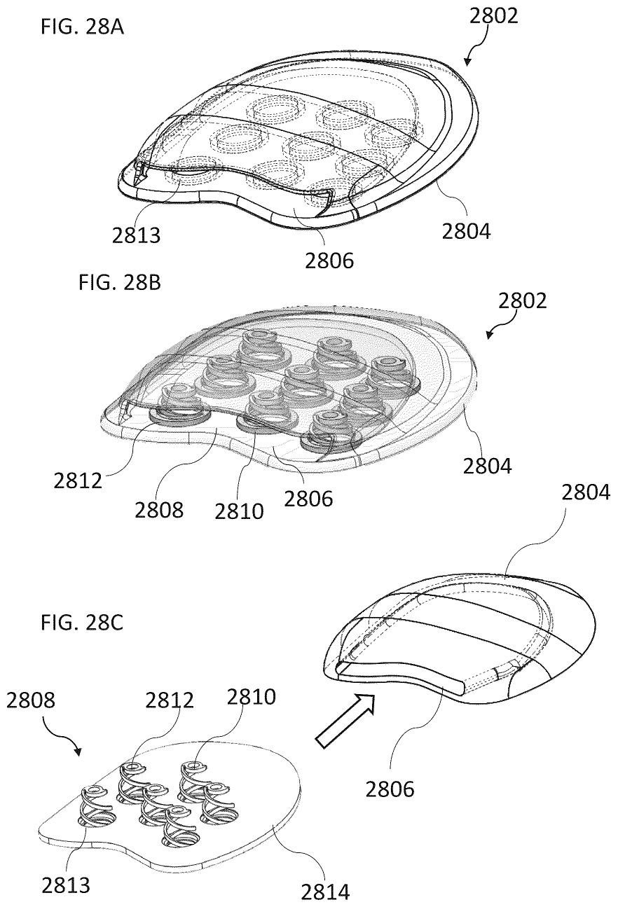

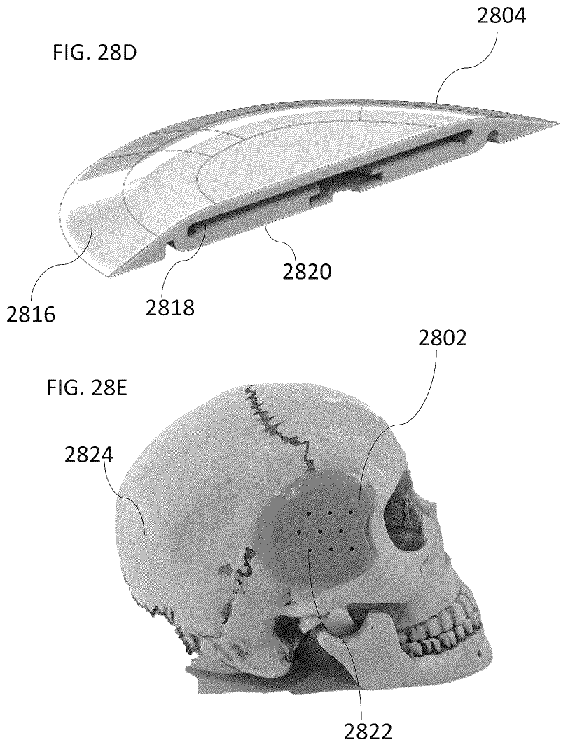

Reference is now made to FIGS. 28A-28D, depicting an implant having an inner actuators array, according to some exemplary embodiments of the invention.

According to some exemplary embodiments, an implant 2802 comprises a chamber 2804 having a wall surrounding and defining an inner lumen. In some embodiments, the chamber 2804 comprises at least one opening 2806 to the inner lumen. In some embodiments, the chamber is formed from an elastic material, and at least one tissue contacting surface of the chamber is soft and/or smooth. In some embodiments, the chamber 2804 is formed as a single unit. Alternatively, the chamber is assembled from two or more separate units coupled to each other.

According to some exemplary embodiments, for example as shown in FIG. 28B, the implant 2802 comprises an inner actuators array 2808. In some embodiments, for example as shown in FIG. 28C, the actuators array 2808 comprises a base 2814 and at least one actuator or a plurality of actuators, for example the actuators 2812 and 2810 coupled to the base 2814, for example using at least one adaptor, at least one lock, using an adhesive and/or a fastener.

According to some exemplary embodiments, the actuators array 2808 is formed by positioning one or more actuators, for example actuators 2810 and 2812 at predetermined actuators positioning locations in the base or on at least one surface of the base. In some embodiments, the base comprises one or more grooves or sockets at each of the positioning locations, for example groove 2813 shown in a transparent view of the implant in FIG. 28A. In some embodiments, the grove 2813 is shaped and sized to receive at least a portion of an actuator, for example an actuator base. In some embodiments, the shape of the groove matches a shape of the actuator portion, for example the actuator base. In some embodiments, the shape of the groove is circular, round, elliptical, quadrilateral, shape of a polygon, or triangle or any geometrical shape that matches the portion of the actuator configured to be positioned inside the groove 2813.

According to some exemplary embodiments, in an implant, one or more of the grooves are empty. In some embodiments, the grooves are spaced-apart, and have a minimal distance between 0.1 mm and 10 mm between each other, for example a minimal distance between 0.1 mm and 1 mm, between 0.5 mm and 3 mm, between 0.5 mm and 5 mm or any intermediate, smaller or larger distance or range of distance values.

According to some exemplary embodiments, for example as shown in FIG. 28C, the implant 2802 is assembled, for example, by inserting the actuators array 2808 into the chamber lumen via the at least one opening 2806. In some embodiments, following insertion, the actuators array is coupled to the chamber, for example to an inner surface of the chamber wall, using adhesive, or one or more pins or staples penetrating at least partly through chamber 2804 and the array 2808.

According to some exemplary embodiments, for example as shown in FIG. 28D, the chamber 2804 has a tapered edge 2816 surrounding at least partly the chamber 2804. In some embodiments, the chamber 2804 comprises an inner lumen 2818, surrounded by wall 2820.

According to some exemplary embodiments, for example as shown in FIG. 28E, the implant, for example implant 2802 comprises one or more holes or openings in the chamber, for example openings 2822. In some embodiments, the openings are configured to allow penetration of cells during a healing process into the implant lumen, for example lumen 2818. In some embodiments, for example as shown in FIG. 28E, the implant 2802 is used in temporal skull surgeries, as a temporal implant. In some embodiments, the implant is placed in a temple area of a skull 2824, and is used to create volume between the skull and the skin. Alternatively, the implant is implanted at a Chin region of the face, a Cheek region of the face, a Jaw region of the face or any other region in the face or head.

A potential advantage of having an implant formed from an enclosed flexible compartment with an array of actuators within the compartment lumen, for example as shown in FIG. 28B may be to have a more unified design where the actuators are kept inside the chamber and are not in direct contact with tissue, which will optionally lead to longer tissue growth around the implant and longer periods of implant adjustment post surgery. An additional advantage may be to allow easy extraction of the implant with the actuators as a single unit from the body, if needed.

It is expected that during the life of a patent maturing from this application many relevant ACTUATORS will be developed; the scope of the terms actuator or actuators is intended to include all such new technologies a priori.

As used herein with reference to quantity or value, the term “about” means “within ±10 % of”.

The terms “comprises”, “comprising”, “includes”, “including”, “has”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular forms “a”, “an” and “the” include plural references unless the context clearly dictates otherwise. For example, the term “a compound” or “at least one compound” may include a plurality of compounds, including mixtures thereof.

Throughout this application, embodiments of this invention may be presented with reference to a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as “from 1 to 6” should be considered to have specifically disclosed subranges such as “from 1 to 3”, “from 1 to 4”, “from 1 to 5”, “from 2 to 4”, “from 2 to 6”, “from 3 to 6”, etc.; as well as individual numbers within that range, for example, 1, 2, 3, 4, 5, and 6. This applies regardless of the breadth of the range.

Whenever a numerical range is indicated herein (for example “10-15”, “10 to 15”, or any pair of numbers linked by these another such range indication), it is meant to include any number (fractional or integral) within the indicated range limits, including the range limits, unless the context clearly dictates otherwise. The phrases “range/ranging/ranges between” a first indicate number and a second indicate number and “range/ranging/ranges from” a first indicate number “to”, “up to”, “until” or “through” (or another such range-indicating term) a second indicate number are used herein interchangeably and are meant to include the first and second indicated numbers and all the fractional and integral numbers therebetween.

Unless otherwise indicated, numbers used herein and any number ranges based thereon are approximations within the accuracy of reasonable measurement and rounding errors as understood by persons skilled in the art.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1. A method for shaping tissue, comprising:
providing an implant comprising at least one actuator configured to expand and/or contract when exposed to an external energy;
implanting the implant into an implantation site in a patient body between at least one first tissue and at least one second tissue of the body;
selectively energizing the at least one actuator, wherein when said at least one actuator is selectively energized, said at least one actuator applies force on said at least one first tissue and/or on said at least one second tissue;
shaping said at least one first tissue and/or said at least one second tissue according to said applied force.

2. A method according to claim 1, wherein said selectively energizing comprises remotely selectively energizing said at least one actuator from a remote location outside the body, wherein said selectively energizing comprises selectively heating said at least one actuator to a temperature level that causes said at least one actuator to expand.

3. (canceled)

4. A method according to claim 2, wherein said selectively heating comprises exposing said at least one actuator to one or more of, an electromagnetic field generated outside the body, ultrasound energy, radiofrequency energy, laser, infra-red energy, or warm liquid.

5. (canceled)

6. A method according to claim 1, wherein said selectively energizing is performed prior to, during and/or following said implanting.

7. A method according to claim 1, comprising allowing said implantation site to heal prior to said selectively energizing.

8. A method according to claim 1, comprising repeating said selectively energizing and said shaping if said shaped tissue does not acquire a target shape.

9. A method according to claim 1, wherein said providing comprises providing said implant with at least one tissue interface, and wherein said at least one actuator is coupled to said at least one tissue interface, and wherein said implanting comprises placing in contact said at least one actuator with said at least one second tissue and placing in contact said at least one tissue interface with said at least one first tissue.

10. A method according to claim 1, wherein said implanting comprises plastic or elastic bending said implant to conform to a surface of said at least one first tissue or to a surface of said at least one second tissue.

11-12. (canceled)

13. A method according to claim 1, wherein said at least one first tissue comprises soft tissue, and wherein said at least one second tissue comprises bone tissue, and wherein said shaping comprises shaping said soft tissue according to expansion of said energized at least one actuator.

14-15. (canceled)

16. A body implant configured to be implanted within an implantation site in a body, comprising:
a body, configured to move between a collapsed state and an expanded state, wherein said body comprising:
at least one actuator shaped and sized to contact body tissue;
a plurality of actuators coupled to said at least one actuator base, wherein at least one actuator of said plurality of actuators is configured to contractapply force against said at least one actuator base and said body tissue when exposed to energy.

17. A body implant according to claim 16, wherein said plurality of actuators comprise apertured actuators.

18-19. (canceled)

20. A body implant according to claim 16, wherein at least some of said plurality of actuators are coupled to each other by one or more connectors.

21. A body implant according to claim 16, wherein when heated by said energy, at least some of said plurality of actuators are configured to move laterally relative to each other and/or in a direction substantially perpendicular to said base or a direction oriented at an angle between 10 degrees and 170 degrees relative to said base

22-24. (canceled)

25. A body implant according to claim 16, wherein each of said plurality of actuators comprises a spring formed from a shape memory material, that is configured to expand when heated by said energy, wherein said spring is shaped as a spiral, or a helix.

26-36. (canceled)

37. A body implant according to claim 16, wherein a thickness of said body in said collapsed state is within a range between 1 mm and 4 mm.

38-55. (canceled)

56. A method according to claim 1, wherein said selectively energizing comprises energizing said at least one actuator with energy parameters suitable for changing a shape of said at least one actuator.

57. A method according to claim 1, wherein when said at least one actuator is selectively energized, an expansion state of said at least one actuator is modified, and wherein said shaping comprises shaping said at least one first tissue and/or said at least one second tissue according to said modified expansion state of said at least one actuator.

58. A body implant according to claim 16, wherein said body comprising a tissue contacting interface configured to contact body tissue, wherein said tissue contacting interface is soft and/or flexible, and wherein said at least one actuator is configured to apply force on said tissue contacting interface when exposed to said energy.

59. A body implant according to claim 58, wherein said body comprises one or more perforations which are shaped and sized to allow tissue ingrowth and/or injection of fluid, into said body implant.

60. A body implant according to claim 16, wherein each of said plurality of actuators comprises a shape memory material, configured to expand or contract when heated by said energy.
